(19) United States
(12) Patent Application Publication
(10) Pub. No.: US 2023/0282316 A1
(43) Pub. Date: Sep. 7, 2023

(54) SYSTEMS AND METHODS FOR EMISSION SOURCE ATTRIBUTION

(71) Applicant: Microsoft Technology Licensing, LLC, Redmond, WA (US)

(72) Inventors: Sara MALVAR MAUA, Sao Paulo (BR); Leonardo DE OLIVEIRA NUNES, Rio de Janeiro (BR); Mirco MILLETARI', Singapore (SG); Neera Bansal TALBERT, Houston, TX (US); Yazeed Khalid ALAUDAH, Houston, TX (US); Jeremy Randall REYNOLDS, Boulder, CO (US); Yagna Deepika ORUGANTI, Houston, TX (US); Ashish BHATIA, Cambridge, MA (US); Anirudh BADAM, Issaquah, WA (US)

(73) Assignee: Microsoft Technology Licensing, LLC, Redmond, WA (US)

(21) Appl. No.: 17/807,685

(22) Filed: Jun. 17, 2022

Related U.S. Application Data
(60) Provisional application No. 63/364,022, filed on May 2, 2022.
(60) Provisional application No. 63/315,486, filed on Mar. 1, 2022.

(51) Int. Cl. G16C 20/70; G16C 20/80; G06N 20/20; G06N 7/00

(52) U.S. Cl. CPC G16C 20/70; G06N 7/005; G06N 20/20; G16C 20/80

(57) ABSTRACT

A method for source attribution comprises receiving measurements of a chemical species at a spatially distributed sensor array for a given set of spatially positioned emission sources in a physical environment using a dispersion model. Based on the received measurements, a concentration field is mapped from the emission sources to the sensor array using a forward operator. For each emission source, a likelihood data set is evaluated at least by fitting an emission rate of the chemical species using a regression model based on the mapped concentration field and real-world, runtime measurements from the sensor array. A posterior data set is evaluated based at least on the evaluated likelihood data set and historical data for the physical environment. For each sensor of the sensor array, estimated emission rates and contribution rankings for emission sources are determined and output based on the evaluation of the posterior data set.

### CROSS REFERENCE TO RELATED APPLICATIONS

This application claims priority to U.S. Provisional Pat. Application Ser. No. 63/315,486, filed Mar. 1, 2022, and to U.S. Provisional Pat. Application Ser. No. 63/364,022 filed May 2, 2022, the entirety of each of which is hereby incorporated herein by reference for all purposes.

### BACKGROUND

Emissions of a chemical species within a physical environment may take various forms, including gases, aerosols, particulate, or other fluids. Examples of atmospheric emissions include methane leaks from industrial infrastructure or products of combustion such as smoke from combustion sources. Emissions into liquid environments, such as a body of water, may take liquid form, such as petroleum, as an example.

Sensors may be used to monitor a physical environment for the presence of a chemical species. Sensor measurements may enable emission sources to be identified and quantified. For example, sensors capable of detecting a concentration of airborne methane gas may be positioned at various locations within a geographic region to enable the geographic region to be monitored for emission of methane into the physical environment. Once emission sources are identified, remedial action can be initiated to quell the leaks.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

A method for source attribution comprises receiving measurements of a chemical species at a spatially distributed sensor array for a given set of spatially positioned emission sources in a physical environment using a dispersion model. Based on the received measurements, a concentration field is mapped from the emission sources to the sensor array using a forward operator. For each emission source, a likelihood data set is evaluated at least by fitting an emission rate of the chemical species using a regression model based on the mapped concentration field and real-world, runtime measurements from the sensor array. A posterior data set is evaluated based at least on the evaluated likelihood data set and historical data for the physical environment. For each sensor of the sensor array, estimated emission rates and contribution rankings for emission sources are determined and output based on the evaluation of the posterior data set.

### BRIEF DESCRIPTION OF THE DRAWINGS

FIG. 1 is a schematic diagram illustrating aspects of a concentration field of a chemical species emitted by multiple independent sources, according to an example.

FIG. 2 is a schematic diagram depicting an example of a computing environment, including a computing system of one or more computing devices.

FIG. 3 is a schematic diagram depicting an example source attribution module.

FIG. 4 is a flow diagram depicting an example source attribution method.

FIG. 5 depicts an aerial view of an example region of interest within a physical environment, showing example locations of emission sources and sensors.

FIGS. 6A and 6B depict an example wind speed and angle distribution.

FIG. 7A depicts an example sample from an emission rate distribution, together with median and bulk.

FIG. 7B depicts measurements at sensor locations.

FIG. 8A depicts an example marginal posterior distribution in which convergence has been achieved.

FIG. 8B depicts an additional example marginal posterior distribution in which the sample mean lies in the tail of the distribution.

FIG. 9 depicts an example network map for the example region of interest of FIG. 5 in which connections between sensors and sources are used to visualize attribution.

FIG. 10A is a graph depicting an example of true and predicted source contributions to a measurement of concentration of a chemical species at a sensor.

FIG. 10B is a graph depicting an example of true and predicted emission rate estimates for the highest three emission sources at a sensor.

FIG. 11 is a schematic architecture diagram showing aspects of the configuration and operation of a source attribution decision machine.

FIG. 12 is a flow diagram depicting an example method for a source attribution decision machine.

FIG. 13 is a plot diagram showing illustrative output generated by an example source attribution decision framework.

FIG. 14 schematically shows an area of influence approach to localizing sources of chemical species.

FIG. 15 schematically shows an example computing device.

### DETAILED DESCRIPTION

In some scenarios, multiple emission sources of a chemical species positioned at different physical locations may contribute to a compound measurement at a sensor. For example, if there are multiple sources of emission within a physical environment, a sensor may measure a compound signal that reflects a combination of emissions from the multiple sources. This compound signal may present challenges to identifying and quantifying emission sources, particularly for underdetermined systems wherein there exist more or many more sources than sensors.

Source attribution problems involving compound measurements belong to a class of problems known as “inverse problems.” Inverse problems can be highly complex and difficult to solve, with non-unique solutions. The uncertainty of estimates, especially in the case of underdetermined systems, can be large. As a result, previous solutions to these problems can consume large amounts of computing resources such as processor cycles, memory, power, etc., and can be time consuming to implement. Additionally, previous solutions may not be operable or effective at source attribution within underdetermined. It is with respect to these and other technical challenges that the disclosure made herein is presented.

According to an aspect of the present disclosure, sources of emission of a chemical species may be identified and quantified by a variety of computer-implemented source attribution techniques and methodologies. As an example, the source attribution techniques disclosed herein may enable decomposition of a compound measurement obtained by one or more sensors from multiple sources of emission, thereby enabling individual emission sources to be identified and quantified among a set of emission sources. A predetermined number of highest-ranking emissions sources may be output, allowing for remedial or corrective action to be performed with respect to particular emission sources, such as sources that emit a chemical species beyond a threshold rate.

In some examples, a Bayesian source attribution approach may be implemented to identify and/or quantify sources of emissions based on sensor measurements obtained within a physical environment. As an example, a physical model, such as a dispersion model, may be used to simulate measurements at multiple sensors of a spatially distributed sensor array for a given set of sources of emission of a chemical species. A forward operator of the physical model, may act on a concentration field of the chemical species from emission sources to sensors of the sensor array to produce a new field configuration. A regression model (e.g., a non-linear Bayesian regression model) may be used to fit an emission rate of the chemical species for each source based on the forward operator and real-world, runtime measurements from sensors of the sensor array, enabling emission sources to be identified and/or quantified even in the context of compound measurements from multiple sources of emission. Feedback from the regression model may be used within the physical model to reduce error and improve accuracy or precision of the implemented source attribution techniques. Within a Bayesian context, the source attribution techniques disclosed herein may represent values being input, processed, or output as a statistical distribution of likelihood. Identification of emission sources and/or quantification of their emissions may be provided as a ranking that is based on a statistical distribution, in at least some examples.

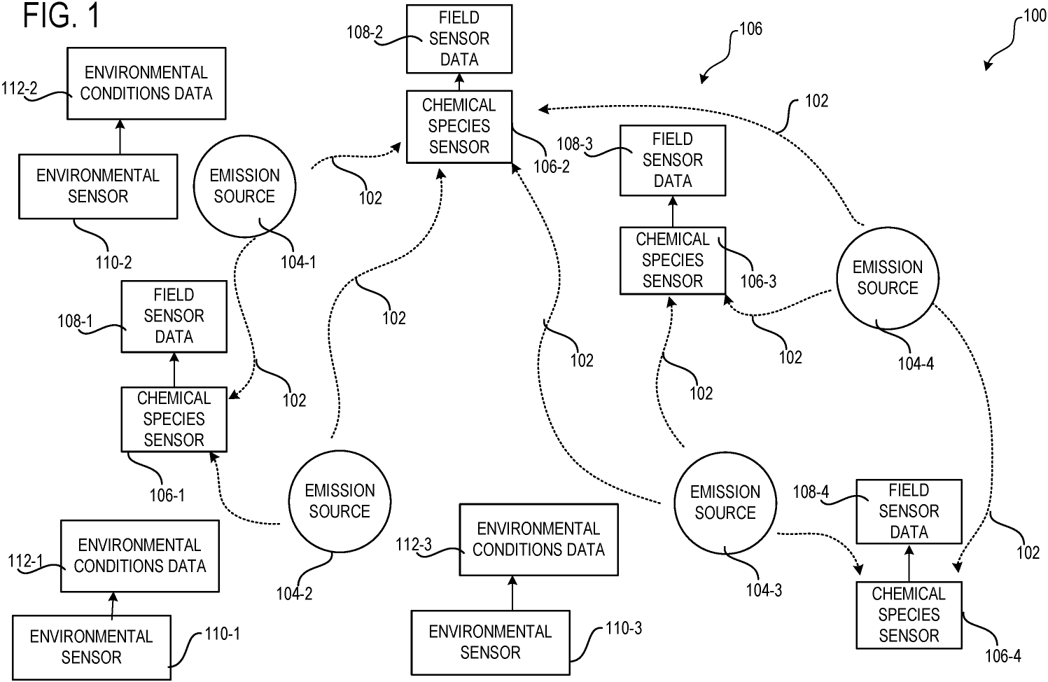

FIG. 1 is a schematic diagram illustrating aspects of physical environment 100 including a concentration field 102 of a chemical species emitted by multiple spatially positioned emission sources (104-1, 104-2, 104-3, 104-4), according to an example. A spatially distributed sensor array 106 includes a plurality of chemical species sensors (106-1, 106-2, 106-3, 106-4) situated at different physical locations within physical environment 100, each sensor capable of detecting the presence and/or concentration of the chemical species at its physical location. Emissions of a chemical species, such as methane, by spatially positioned emission sources (104-1, 104-2, 104-3, 104-4) may contribute to concentration field 102. As such, concentration field 102 may contribute to a compound measurement at a sensor (106-1, 106-2, 106-3, 106-4).

Sensors 106-1, 106-2, 106-3, and 106-4 may thus measure a compound signal that reflects a combination of concentrations of the chemical species generated by some or all of emission sources 104-1, 104-2, 104-3, and 104-4. For instance, in the illustrated example, field sensor data 108-1 generated by sensor 106-1 reflects the concentration of emissions generated by sources 104-1 and 104-2, sensor data 108-2 generated by sensor 106-2 reflects the concentrations of emissions generated by sources 104-1, 104-2, 104-3, and 104-4, sensor data 108-3 generated by sensor 106-3 reflects the concentrations of emissions generated by sources 104-3 and 104-4, and sensor data 108-4 generated by sensor 106-4 reflects the concentrations of emissions generated by sources 104-3 and 104-4. In this regard, it is to be appreciated that many more chemical species sensors and emission sources may be present in a real-world application.

In scenarios such as those described above, it may be desirable to decompose the recorded compound signal to determine the contribution of each of the emission sources 104-1, 104-2, 104-3, and 104-4 contributing to the compound signal. For instance, given field sensor data 108-1, 108-2, 108-3, and 108-4, specifying measurements taken by sensors 106-1, 106-2, 106-3, and 106-4 at various positions in space and time, it may be desirable to determine the probable contribution of each of the emission sources 104-1, 104-2, 104-3, and 104-4 to the compound signal, including the strength of the contribution of each source 104-1, 104-2, 104-3, and 104-4 and the location of each source 104-1, 104-2, 104-3, and 104-4.

Aside from chemical species sensors of spatially distributed sensor array 106, physical environment 100 may include a plurality of environmental sensors (e.g., 110-1, 110-2, and 110-3) configured to measure and record environmental conditions data (e.g., 112-1, 112-2, and 112-3) within physical environment 100 over time. In particular, environmental sensors 110-1, 110-2, and 110-3 may be configured to measure and record conditions which may impact concentration field 102, such as wind velocity, humidity, air temperature, precipitation, seismic activity, air composition, etc. For aquatic physical environments, such as oceans, appropriate sensors, such as water temperature sensors, current speed sensors, microbial sensors, etc. may be used. Environmental sensors may be spatially positioned throughout physical environment 100. In some cases, one or more environmental sensors may be adjacent to, nearby, or coincident with an emission source or emission sensor. In some examples, geographic constraints and parameters physical environment 100 may inform positioning of the environmental sensors.

Once the probable contribution of each of the sources 104-1, 104-2, 104-3, and 104-4 has been determined, remedial or corrective action may be taken such as, for example, dispatching maintenance crews to investigate and address potential leaks of the chemical species. If potential leaks are located, the maintenance crews can stop or reduce the emission of the chemical species from one or more of the sources, which may include the sources identified as having the greatest emission of the chemical species.

To accurately parse the contribution of each emission source to the field sensor data collected by the chemical species sensor of the spatially positioned sensor array, field sensor data, environmental conditions data, and other data may be evaluated by one or more source attribution models in the context of a computing environment, for example.

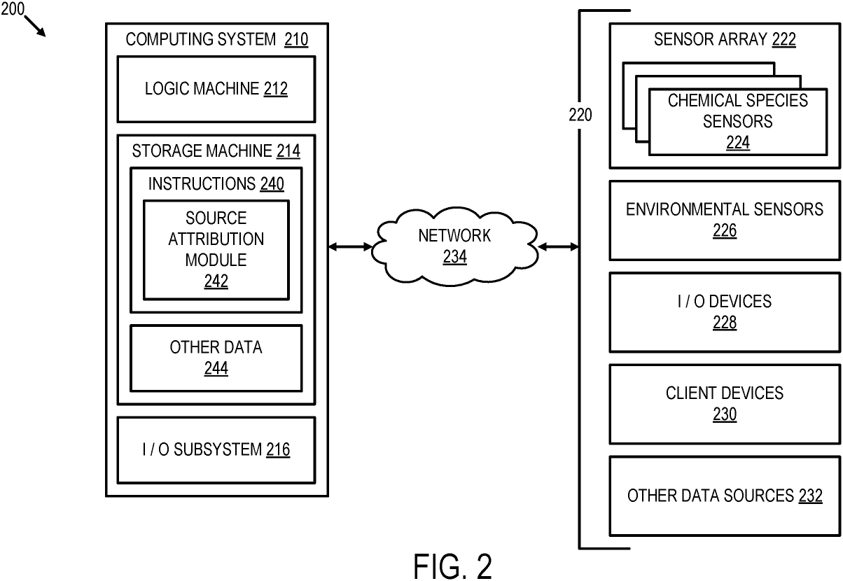

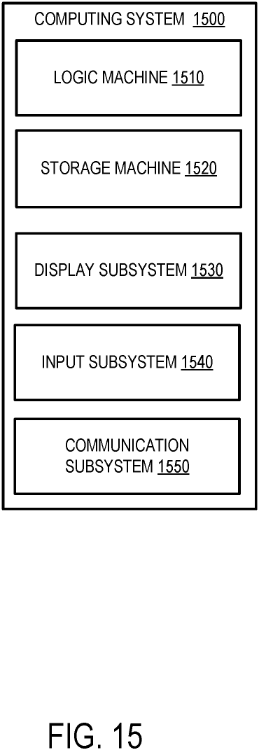

FIG. 2 is a schematic diagram depicting an example of a computing environment 200, including a computing system 210 of one or more computing devices. Computing system 210 is depicted in FIG. 2 in simplified form. As an example, computing system 210 includes a logic machine 212, a storage machine 214, and an input / output subsystem 216 by which computing system 210 may communicate with one or more other devices and/or sources of data, identified schematically at 220. A more detailed description of an exemplary computing system is described herein and with regard to FIG. 15.

Examples of other devices and/or sources of data identified at 220 include a spatially distributed sensor array 222 of multiple chemical species sensors 224 that measure a concentration of a chemical species, one or more environmental sensors 226, that measure environmental conditions such as wind velocity (e.g., magnitude and direction), one or more input and/or output devices 228 by which users or other peripheral devices may interact with computing system 210, one or more client devices 230 which may include other computing devices or computing systems, and other data sources 232. In at least some examples, devices and/or sources of data identified at 220 may communicate with computing system 210 via a communications network 234.

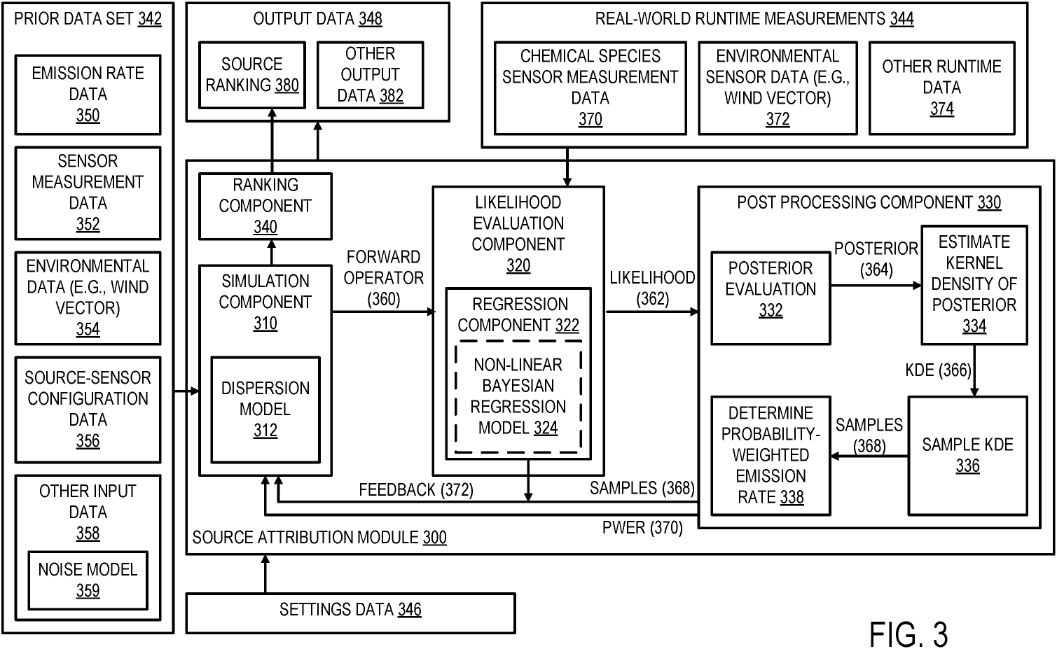

Storage machine 214 includes instructions 240 stored thereon executable by logic machine 212 to perform the various computer-implemented operations and processes disclosed herein. As an example, instructions 240 may include or otherwise define a source attribution module 242, an example of which is described in further detail with reference to FIG. 3. Storage machine 214 may include other data 244 stored thereon, including data input to, processed by, and output from computing system 210, as examples. Sensor array 222 and environmental sensors 226 may be configured to provide real-world run-time data (e.g., current or near real-time data) to computing system 210, either directly or indirectly (e.g. following pre-processing). Such run-time data may also be processed, stored, and/or archived on one or more storage devices as historical or prior data that may be provided to source attribution model 244.

As previously discussed, source attribution, which may also be referred to as source retrieval, belongs to the class of inverse problems. An example aim of source attribution is to identify one or more sources of emission that generated a certain concentration field of a chemical species given measurements of field values (e.g., by sensors) at a set of points {x₁, x₂, . . ., x_{M}} ∈ R^{d}, where d is the number of space dimensions.

During some observation time δt, some or all the sensors deployed in the physical environment may record concentration measurements of the chemical species. As there may be multiple sources being monitored in the physical environment, the sensors may each record a compound measurement that is assumed to be given by a linear combination of concentrations generated by a subset of sources (at least some of the sources) at each sensor location. An example objective may be to find the decomposition of the compound measurement to determine the contribution of each source to the measured concentration. As an example, a set of k sources may be identified for each sensor that contribute to the compound measurement of the sensor, and an emission rate of those sources may be quantified. In source attribution, emission rates of the N sources q = [q₁, q₂, . . ., q_{N}]^{T} may be unknown. Emission rate may be measured in mass per unit time (e.g., kg / h, kg / s, etc.), as an example.

FIG. 3 is a schematic diagram depicting an example source attribution module 300. Source attribution module 300 is an example of previously described source attribution module 242 of FIG. 2, which may be executed or otherwise implemented by a computing system, such as example computing system 210.

Source attribution module 300 may include one or more components. In this example, source attribution module 300 includes at least a simulation component 310, a likelihood evaluation component 320, a post processing component 330, and a ranking component 340. Source attribution module 300 may include additional components, as described in further detail herein.

Source attribution module 300 obtains a variety of input data, which may include prior data set 342, real-world runtime measurements 344, settings data 346, etc. Based on the input data, source attribution module 300 generates output data 348, which may include identification and/or quantification of emissions of a chemical species of one or more emission sources. As an example, output data 348 generated by source attribution module 300 includes a source ranking 380 generated by ranking component 340, and/or other output data 382.

Prior data set 342 may include emission rate data 350 of sources, sensor measurement data 352 of sensors, weather data 354 (e.g., wind vector and/or other forms of weather data described herein), source-sensor configuration data 356, and other input data 358. Prior data set 342, in at least some implementations, may be obtained and used by source attribution module 300 as part of a model development phase. This model development phase may proceed real time or near-real time processing of real-world, runtime measurements 344 used for source attribution, in at least some implementations. Simulation component 310 may use prior data set 342 in combination with settings data 346 as input to a dispersion model 312 to generate simulated data, including a forward operator 360 which may be provided to likelihood evaluation component 320.

As examples, emission rate data 350 may include historical data and/or assumption-based data of measurements of emission rate of a chemical species at each source of a set of multiple sources within a physical environment. Sensor measurement data 352 may include historical data of measurements of concentration of a chemical species at each sensor of a sensor array within the physical environment, if available.

Weather data 354 may include historical data and/or assumption-based data of measurements of one or more wind vectors and/or other forms of weather data. As an example, wind vectors having a magnitude and direction may be measured within the physical environment by one or more wind sensors located within that physical environment. Alternatively or additionally, weather data 354 may be obtained from third-party sources.

Source-sensor configuration data 356 may define a spatial configuration of the multiple sensors of the sensor array and of the multiple sources of the chemical species spatially distributed within the physical environment. As an example, a location (e.g., in three-dimension space) of each sensor and each source may be defined within a three-dimensional coordinate system. Other input data 358 may include physical properties (e.g., a density) of the chemical species, physical properties (e.g., a density) of a medium (e.g., air, water, etc.) within which the chemical species disperses, and other suitable data that may form part of prior knowledge 304, as described herein.

Settings data 346 used by source attribution module 300 may include data that defines one or more values of parameters that can be varied or tuned by a user, examples of which may include a confidence interval, a highest posterior density interval (HPDI) for sampling of statistical distributions, measurement thresholds for concentrations of the chemical species as measured by sensors to initiate source attribution processes, and other suitable settings described herein.

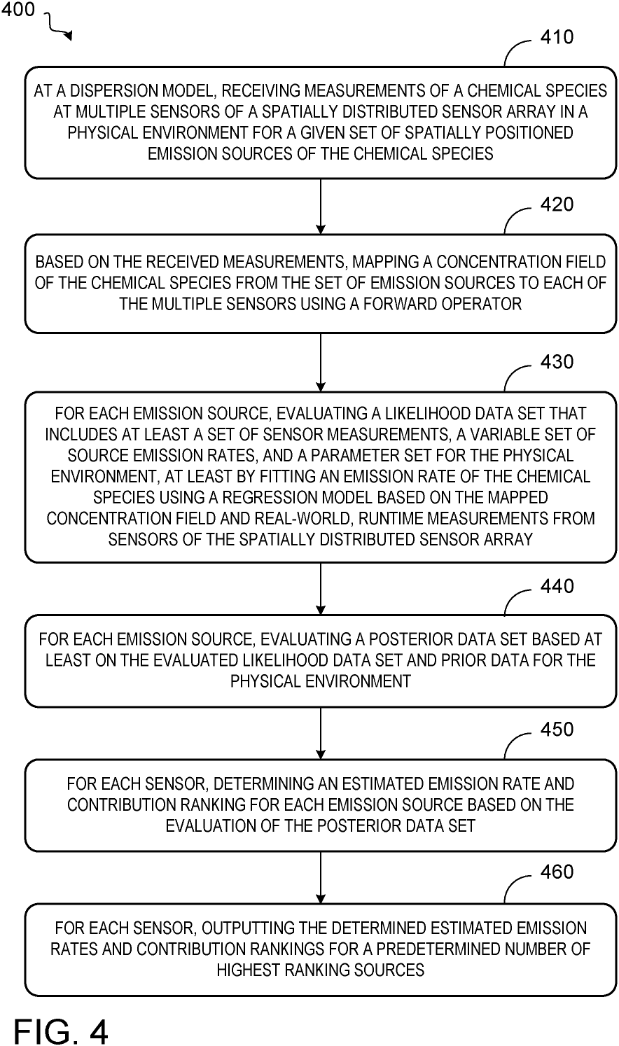

Turning to FIG. 4, a flow diagram depicting an example computer-implemented method 400 for source attribution is shown. Method 400 may be performed by a computing system, such as example computing system 210 of FIG. 2. As an example, method 400 may be performed by computing system 210 executing source attribution module 242, an example of which includes source attribution module 300 of FIG. 3.

At 410, method 400 includes, at a dispersion model, receiving measurements of a chemical species at multiple sensors of a spatially distributed sensor array in a physical environment for a given set of spatially positioned emission sources of the chemical species.

The method may include obtaining source-sensor configuration data defining a spatial configuration of the multiple sensors of the sensor array and of multiple sources of the chemical species spatially distributed within the physical environment. An example of the source-sensor configuration data includes data 356 of FIG. 3, as an example.

The method may further include obtaining a prior data set (P(q, θ)) (e.g., prior data set 342 of FIG. 3) representing a distribution of a background emission rate of each source of the multiple sources and a distribution of wind velocity for the physical environment.

The dispersion model, e.g., dispersion model 312, may then simulate a distribution of sensor measurements at each sensor based on the prior data set and the source-sensor configuration data.

In some examples, the dispersion model is a physics-based dispersion model. For example, simulation component 310 may implement a physics-based dispersion model 312 to simulate measurements of a chemical species at multiple sensors of a spatially distributed sensor array for a given set of sources of emission of the chemical species. As examples, dispersion model 312 may take the form of a Gaussian plume dispersion model, weather research and forecasting coupled with chemistry (WRF-CHEM), an integrated Lagrangian puff modeling system (e.g., CALPUFF), etc.

In some examples, receiving measurements of the chemical species at multiple sensors of the spatially distributed sensor array in the physical environment for the given set of spatially positioned emission sources of the chemical species includes generating a distribution of sensor measurements based on a prior data set representing a distribution of a background emission rate of each source and a distribution of wind velocity in the physical environment.

For example, dispersion model 312 may be used to model or otherwise simulate dispersion of a chemical species within a physical environment using emission rate of an emission source, wind vector, position of the emission source (e.g., height), and other suitable input data. As an example, plume evolution for a chemical species over time (e.g., over a 24 hour period or other suitable period of time) may be modeled or otherwise simulated by dispersion model 312 based on input data in the form of prior data set 342.

In at least some examples, to accelerate the dispersion model (e.g., Gaussian plume model) for large scale simulations, evaluation over both sensors and sources may be parallelized (e.g., by source-sensor pairs). As an example, a machine learning framework (e.g., PyTorch) may be used to parallelize evaluations over both sensors and sources. Parallelized evaluation may provide an order of magnitude or greater increase in processing speed, as an example. Additionally, graphics processing units (GPUs) may be used to perform parallelized evaluation to further increase processing speed over general purpose processors. Moreover, this enables gradient evaluation of training parameters in the model (e.g., emission rates and/or atmospheric data) for the Bayesian optimization process using e.g., - the Hamiltonian Monte Carlo algorithm (e.g., within the Bayesian optimization library, Pyro).

Returning to FIG. 4, at 420, method 400 includes, based on the received measurements, mapping a concentration field of the chemical species from the set of emission sources to each of the multiple sensors using a forward operator. In at least some examples, there may be more or many more sources N than sensors M such that M << N, presenting an underdetermined system. The forward operator mapping Aₘₙ may take the form of a non-linear, time dependent mapping. The concentration field of the chemical species may be mapped from the distribution of the baseline emission rate at the source of a source-sensor pair to the distribution of the received measurement at the sensor of the respective source-sensor pair.

For each source-sensor pair of source-sensor configuration data 356, a forward operator 360 may be generated by simulation component 310 based on an output of the physical model (e.g., dispersion model 312) that maps a concentration field of the chemical species from the source of the source-sensor pair to the sensor of the source-sensor pair for a given time step. As an example, a respective forward operator 360 may be generated for each source-sensor pair of source-sensor configuration data 356. As an illustrative example, for 100 sources and 15 sensors, a total of 150 source-sensor pairs may be modeled by simulation component 310 for a total of 150 forward operators 360 per time step.

The term Aₘₙ(q, θ) may be referred to as the forward operator mapping of the M x N (e.g., M sensors and N sources) that maps the concentration field from source location to sensor location, parametrized by θ = {u, Φ, p}. In this example, u refers to the modulus of wind velocity (e.g., m / s), Φ refers to an in-plane direction (e.g., radians) of the wind velocity, and p refers to other parameters. Other parameters p (examples of which are described in further detail herein) may or may not be included in parameter set p, depending on implementation. While parameters of parameter set θ include measured values that may include uncertainty, the emission rates q of the n sources are unknown and constitute fitting parameters of the model (e.g., 322).

Returning to FIG. 4, at 430, method 400 includes, for each emission source, evaluating a likelihood data set that includes at least a set of sensor measurements, a variable set of source emission rates, and a parameter set for the physical environment, at least by fitting an emission rate of the chemical species using a regression model based on the mapped concentration field and real-world, runtime measurements from sensors of the spatially distributed sensor array. In some examples, the regression model is a Bayesian regression model, such as a non-linear Bayesian regression model. For example, a likelihood data set (P(w|q, θ)) may be evaluated at a non-linear Bayesian regression model based on measurements from each sensor of the sensor array and weather data by fitting an estimated emission rate of each source as a contribution to the measurement of each sensor. A regression model may be applied to the runtime measurement from the sensor of each source-sensor pair and the wind vector of the runtime environmental data, based on the forward operator to fit an estimated distribution of emission rate of the chemical species at the source of the source-sensor pair.

In at least some examples, a Bayesian approach may be used by source attribution module 300 to identify and/or quantify sources of emission of a chemical species. This Bayesian approach may utilize an inversion of a forward model (e.g., dispersion model 312) using Bayes’ principle and one or more statistical sampling algorithms (e.g., Hamiltonian Monte Carlo and/or other Markov Chain Monte Carlo (MCMC) methods, Stochastic Variational Inference (SVI)), etc.) at post processing component 330.

In a physical model, such as dispersion model 312, empirical parameters may be subject to systematic and/or statistical errors. Systematic errors include measurement error associated with instruments such as sensors. Statistical errors include statistical uncertainty in a set of measurements. According to the Bayesian approach disclosed herein, uncertainty associated with data inputs to a Bayesian model propagate through the model in a non-parametric fashion. As a result, inferred parameters may be associated with confidence levels that better reflect the physical reality of the model. This means that quantities may be expressed by probability distributions rather than an individual values.

Given a parameter set θ, a variable set q, and sensor measurements w for a chemical species, the Bayesian approach disclosed herein may be expressed by the relationship of Equation 1, where P(q, θ) is the prior, P(w|q, θ) is the likelihood, P(q, θ|w) is the posterior, and Z(w) is a normalization. The prior P(q, θ) may be based on knowledge or assumptions on the form of the distribution (e.g., prior data set 342), as an example, and may be communicated to simulation component 310.

$$P \left(q , θ \left|w\right)\right) = \frac{P \left(w \left|q , θ\right)\right) P \left(q , θ\right)}{z \left(w\right)} \qquad \text{(1)}$$

Given an array of M sensors, wₘ represents a compound measurement by sensor m of the sensor array at time interval δt. The time interval δt may refer to a sampling frequency of the sensors, as an example. The compound measurement Wₘ (i.e., compound signal) may be expressed by the relationship of Equation 2. Within Eq. (2), the forward operator of the combined forward operator mappings is represented by the term A(q, θ).

$${w}_{m} = {\sum }_{n = 1}^{N} {A}_{m n} \left({q}_{n} , θ\right) ≡ A \left(q , θ\right) \qquad \text{(2)}$$

In at least some examples, a set of assumptions may be implemented on the form of the concentration field C(x, t) and, therefore, of the output of the forward model (e.g., A(q, θ)) that may make the problem to be solved more numerically manageable at different levels of complexity. As described above, the forward operator mapping Aₘₙ may be based on dispersion model 312 (e.g., a Gaussian plume model) to simulate a source’s contribution to the compound signal measured by a sensor.

In at least some examples, there may be more or many more sources than sensors such that M « N, presenting an underdetermined system. The forward operator mapping Aₘₙ may take the form of a non-linear, time dependent mapping. As an example, the forward operator mapping Aₘₙ may be represented as a solution of the Diffusion-Advection partial differential equation (PDE). The PDE may be expressed by the relationship of Equation 3, where L(θ) is a linear operator that may depend nonlinearly (or linearly depending on implementation) on the parameters θ, comprising an advection, D(x), a diffusion term controlled by the diffusion matrix, and C(x, t), a term that represents the concentration field at location x = (x, y, z) and time t.

$$\begin{matrix}\left({\partial }_{t} + L \left(θ\right)\right) C \left(x , t\right) = {\sum }_{n = 1}^{N} {q}_{n} \left(t\right) δ \left(x - {x}_{n}\right) \\ L \left(θ\right) = ∇ ⋅ \left(u \left(x , t\right) - D \left(x\right) ∇\right)\end{matrix} \qquad \text{(3)}$$

The location in this example is defined within the physical environment in three spatial dimensions x, y, and z (height dimension). The linear operator L(θ) may comprise an advection and a diffusion term controlled by the diffusion matrix, D(x). As an example, source attribution module 300 considers sources as point-emission sources specified by the xₙ coordinates in the Dirac delta function represented by

$$\sum {}_{n = 1}^{N} {q}_{n} \left(t\right) δ \left(x - {x}_{n}\right) .$$

In at least some examples, the forward operator mapping Aₘₙ may be represented as a special solution of the PDE of Eq. (3), under the following simplifying assumptions or conditions: (1) the emission rates q(t) vary slowly over time such that each emission rate can be considered constant over a measurement time interval δt (e.g., q(t) = q); (2) the wind velocity (magnitude and direction) does not change over the measurement time interval δt and the wind velocity is aligned along the x direction for x ≥ 0 - i.e., u = (u, 0, 0); and (3) the diffusion matrix, D(x) is replaced by effective parameters based on the Pasquill stability classes. Additionally, boundary conditions may include finiteness of the concentration field of the chemical species at the origin and infinity, together with the condition that the chemical species does not penetrate the ground surface.

Under the above assumptions or conditions, the PDE admits an analytical solution in the form of a Gaussian kernel that may be represented by the relationship of Equation 4.

$${C}_{n} \left(x\right) = \frac{{q}_{n}}{2 π u {σ}_{y} {σ}_{z}} exp \left\{- \frac{{\left(z - h\right)}^{2}}{2 {σ}_{z}^{2}} - \frac{{\left(z - h\right)}^{2}}{2 {σ}_{z}^{2}} - \frac{{y}^{2}}{2 {σ}_{y}^{2}}\right\} \qquad \text{(4)}$$

In this example, the concentration field C (x, y, z), as a scalar, may be measured in terms of mass per unit volume (e.g., kg / m³). However, it will be understood that the concentration field may be measured in terms of parts per million volume (ppmv). Within the above Gaussian kernel, The σᵢ terms (e.g., σ_{X}, σ_{y}, σ_{z}) represent standard deviations, and the h term represents the height of the source (e.g., in a z direction).

In at least some examples, an implementation of the dispersion model (e.g., a Gaussian plume model) may incorporate a redefined value of the standard deviation terms σi to include heuristic information on the stability of the plume, which may be represented by the relationship of Equation 5, where the values of the parameters ai, bi, cᵢ depend on the atmospheric stability class (indexed from A to F) and are different for the y and z components.

$${σ}_{i} \left(x\right) = {a}_{i} x {\left(1 + x {b}_{i}^{- 1}\right)}^{- {c}_{i}} \qquad \text{(5)}$$

In at least some examples, weather stability classes may be evaluated based on one or more of: wind velocity, cloud coverage, solar activity data, etc. Wind direction and source location may be re-introduced respectively by rotating the simulation grid in-plane and by re-centering the simulation grid on the source position. This expression, linear in the emission rate q may be used as the diffusion operator of a linear regression model.

However, in at least some examples, buoyancy corrections to dispersion along the z axis may additionally be considered, for example, as defined by the relationship of Equation 6, where g is the gravitational constant ρ_{CH4} and ρₐᵢᵣ are the density of methane and air measured in standard conditions.

$$B = \frac{g Q}{π} \left(\frac{1}{{ρ}_{C {H}_{4}}} - \frac{1}{{ρ}_{a i r}}\right) \qquad \text{(6)}$$

It will be understood that the density of methane may be replaced with the density of other chemical species being evaluated, and that the density of air may be replaced with the density of other fluids occupying a physical environment being evaluated. Buoyancy may be measured in units of m⁴ / s³, as an example. Buoyancy may be introduced heuristically in the Gaussian kernel of Eq. (4) by deforming the z-component as shown by the relationship of Equation 7. This transformation introduces a non-linear dependence on emission rate q, making the resulting source retrieval model non-linear.

$${z}^{′} = z + 1.6 \frac{{B}^{1 / 3} {x}^{2 / 3}}{u} \qquad \text{(7)}$$

In scenarios where the number of sensors M is less than or much less than the number of sources N (e.g., M << N), corresponding to a low-density sensor placement, the problem of source attribution may be referred to as being underdetermined. While the parameters θ depend on atmospheric conditions, the emission rates depend on the specifics of the physical process that led to the emission. Therefore, θ and q may be reasonably assumed to be statistically independent of each other. As a consequence, the prior distribution factorizes as shown in Equation 8, where the distribution on θ is obtained via direct measurement of the weather data at a particular location, together with their experimental (and possibly statistical) uncertainties due to temporal or spatial averaging.

$$P \left(q , θ\right) = P \left(q\right) P \left(θ\right) \qquad \text{(8)}$$

A regression model of regression component 322, such as a non-linear Bayesian regression model 324, may be used to fit an estimated emission rate (e.g., an estimated distribution of emission rate) of the chemical species for each source based on the forward operator 360 and real-world runtime measurements (e.g., sensor measurement data 370) from sensors of the sensor array using environmental sensor data 372 and/or other runtime data 374. Likelihood evaluation component 320 obtains each forward operator 360 generated by simulation component 310 and real-world, runtime measurements 344 (and additionally settings data 346 in at least some examples), and generates the likelihood 362 (P(w|q, θ)) via likelihood evaluation component 320. Assuming a normal distribution of the noise with covariance matrix Σ, the likelihood 362 of the model (e.g., evaluated at 320) may be represented by the relationship of Equation 9.

$$P \left(w \left|q , θ\right)\right) = \frac{1}{{\left(2 π det \text{Σ}\right)}^{1 / 2}} {e}^{- \frac{1}{2} {\left‖{\text{Σ}}^{- \frac{1}{2}} \left(w - A \left(q , θ\right)\right)\right‖}^{2}} \qquad \text{(9)}$$

Real-world, runtime measurements 344, in at least some examples, may be obtained and used by source attribution module 300, including likelihood evaluation component 320, as part of a source attribution phase, which may be performed in real time, near-real time, or other suitable time frame, depending on implementation. Real-world, runtime measurements 344 may include sensor measurement data 370 obtained from spatially distributed sensors of the sensor array, indicating concentration of a chemical species within the physical environment from each sensor of the sensor. Real-world, runtime measurements 344 may further include environmental sensor data 372 (e.g., wind vectors having a magnitude and a direction and/or other weather data) from weather sensors within the physical environment or third-party sources, and other runtime data 374. Wind vectors may be obtained for a time range for which the measurement of the concentration of the chemical species was obtained from each sensor of the sensor array.

In at least some examples, a simplifying assumption of θ being deterministic may be used. As an example, ten minute averages or other suitable durations of time of wind vector data may be used over the measurement time of the sensors. This simplifying assumption may be used in scenarios in which the sensors are capable of measuring concentration of the chemical species in real time or near-real time (e.g., within the period of time of the average wind velocity). In the case of methane, for example, sensors may operate in real time or near-real time using direct measurements based on a variety of techniques, such as mid/near infra-red lasers or metal oxide semiconductors, as examples.

In the case of real time or near-real time source attribution, weather data may be taken at the time of measurement of the chemical species by the sensors. In at least some examples, the sensors may include or be associated with weather stations that capture wind velocity and/or other weather data. By taking θ as deterministic, the weather data (e.g., wind velocity, etc.) may be assumed to be homogeneous across the region or volume of interest, in agreement with the same assumption used to obtain the Gaussian plume solution, and neglect systematic errors.

Returning to FIG. 4, at 440, method 400 includes, for each emission source, evaluating a posterior data set based at least on the evaluated likelihood data set and prior data for the physical environment. As an example, post processing component 330 may obtain likelihood 362 as output from likelihood evaluation component 320 and perform posterior evaluation at 332 based on likelihood 362 to generate posterior 364 (P(q, θ|w)). Post processing component 330 may further generate a kernel density estimation (KDE) 366 of the posterior at 334, sample the KDE at 336 to obtain samples 368, and determine a probability-weighted emission rate (PWER) 370 at 338. Samples 368 from the KDE determined at 336 and PWER 370 determined at 338 may be provided as feedback 376 to simulation component 310. Feedback 376 from regression component 322 and/or post processing component 330 may be used by simulation component 310 for ranking and to improve accuracy or precision of the implemented source attribution techniques. For example, the dispersion model may be refined based on feedback from the Bayesian regression model and/or on one or more of the obtained samples and PWER 370.

Continuing at 450, method 400 includes, for each sensor, determining an estimated emission rate and contribution ranking for each emission source based on the evaluation of the posterior data set, for example, a source contribution ranking and a quantification of estimated emission rate at each source of the multiple sources based on the error. As an example, ranking component 340 of FIG. 3 may determine ranking and quantification based on the error.

To rank the source contributions (e.g., via ranking component 340), multi-point estimates of each emission rate’s posterior distribution may be used. As estimators, percentiles from 0 to 100 may be taken at steps of 2 lying in the 68% HPDI confidence interval, plus the sample average. Sampled point estimates may be used to reconstruct the source contribution to the signal measured at each sensor using again the forward model (e.g., dispersion model 312). For each prediction, the error with the observed value at each sensor location may be again evaluated, and the posterior predictive likelihood of Equation 10 may be evaluated.

$${\text{P}}_{s} ≡ {P}_{s} \left(w \left|{q}_{s}^{⋆} , θ\right)\right) , {q}_{s n}^{⋆} \qquad \text{(10)}$$

Within the relationship of Eq. (10), q^{★}ₛₙ the s-th point estimates of the n-th emission rate from the marginal posterior distribution, which will be used in the final ranking step to weight the accuracy/precision of the ranking solution. In this example, the symbol ★ denotes a variable or parameter fixed by a particular operation, e.g. optimization, sorting, or max, as examples.

Each one of the s samples from point estimates (also referred to as point samples) propose a different source reconstruction within the 68% HPDI of the marginal posteriors. By ranking emission sources by their contribution at each sensor, an ensemble of possible ranking may be obtained, which may be represented by the relationship of Equation 11.

$${R}_{m n ⋆}^{s} = arg \text{sort} {A}_{m n}^{s} \left({q}_{s}^{⋆} , θ\right) \qquad \text{(11)}$$

Within Eq.

$${\left(11\right)}^{n} ,$$

s refers to the point sample index and the term

$${A}_{m n}^{s} \left({q}_{s}^{⋆} , θ\right)$$

is the methane concentration (or other chemical species concentration) value of source n measured from sensor m, obtained from the point estimate s of the emission rate.

In at least some examples, each member of the ranking ensemble may be weighted by the related predictive likelihood. The final ranking (e.g., within source ranking 380) may be obtained as a composite estimator in which, for each sensor, the proposed ranking with the highest likelihood may be presented by the relationship of Equation 12.

$${R}_{m {n}^{⋆}} = arg {m a x}_{{\text{P}}^{s}} {\text{P}}_{n}^{s} {R}_{m {n}^{⋆}}^{s} \qquad \text{(12)}$$

To each predicted ranking, a probability may be assigned that is obtained by multiplying the (selected) marginal posterior point estimate of the source and the predictive likelihood as shown by the relationship of Equation 13.

$$P \left({q}_{s}^{*} ∗ , θ \left|w\right)\right) ≅ P \left(w \left|{q}_{s}^{*} ∗ , θ\right)\right) P \left({q}_{s}^{*} ∗\right) \qquad \text{(13)}$$

At 460, method 400 includes, for each sensor, outputting the determined estimated emission rates and contribution rankings for a predetermined number of highest ranking sources. For example, determined estimated emission rates and contribution rankings for a set number (e.g., three) highest ranking sources may be output, rates and rankings for sources within a threshold emission rate of the highest ranking source may be output, rates and rankings for sources with an emission rate a threshold rate above a baseline emission rate may be output, rates and rankings for sources within a threshold distance of the sensor may be output, etc. As an example, the source ranking and quantification may be output via a user interface or may be used in a downstream process implemented by the computing system.

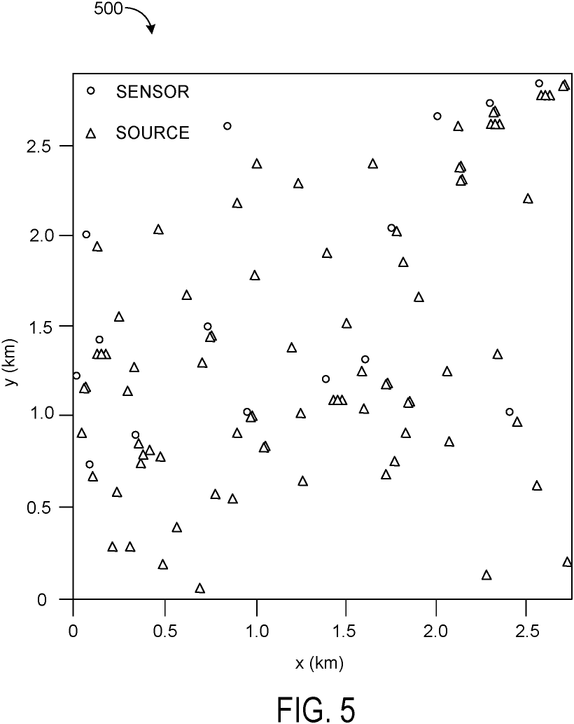

An example implementation of source attribution module 300 is described with reference to a set of 15 spatially distributed sensors of sensor array with respect to a physical environment containing 100 sources of emission of methane. In this example, the 15 sensors and 100 sources are located within a geographic region having an area of approximately 9 km². FIG. 5 depicts a schematic view of an example region of interest, showing example locations of emission sources and sensors based on an aerial view of the region of interest. Within FIG. 5, 100 example emission sources are represented by dark-colored triangles and 15 example sensors for measuring methane are represented by light-colored circles.

An anomaly detection algorithm, described in further detail herein, may be employed to detect large methane emissions; if anomalous readings are detected, these large methane emissions may be flagged to source attribution module 300 that may then return the most likely location(s) of the emissions.

Prior knowledge as input data may be gathered for the physical environment, including environmental data (e.g., 354), historical methane emission rate data (e.g., 350), and oil and gas facility maps that may be used to generate source-sensor configuration data 356. As an example, hourly weather data (wind velocity and air temperature, pressure, cloud coverage, etc.) may be obtained from a weather station within or closest to the physical environment. As another example, methane emissions data can be obtained from aerial surveys, source-located sensor measurements, and/or from historical knowledge of leaks from specific sources.

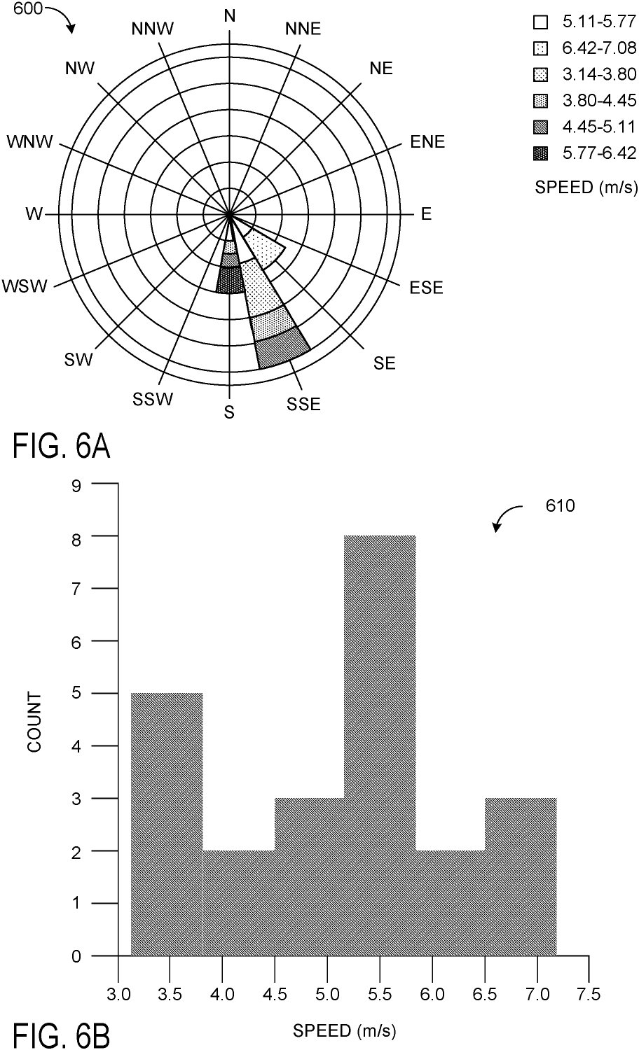

FIGS. 6A and 6B depict an example wind speed and angle distribution for the region of interest of FIG. 5 over a 24-hour period. FIG. 6A depicts the wind speed and angle distribution as a wind rose 600. FIG. 6B depicts the wind speed and angle distribution as a histogram 610.

Given the input data such as prior data set 342, a forward model such as dispersion model 312 may be used to simulate the methane concentration at each sensor location. In this example, the simulation is performed on the region of interest of approximately 9 km² and up to 200 m in the vertical (z) direction. For the simulation, a grid size (dx, dy, dz) of (25, 25, 5) meters is used.

The emission rate of each source may be sampled from the fitted exponential emission rate distribution and weather data at the time of detection is used as an input to a Gaussian plume model, as an example of dispersion model 312. As there are no interaction terms in first expression of Equation 3 that contain the Dirac delta function represented by

$${\sum }_{n = 1}^{N} {q}_{n} \left(t\right) δ \left(x - {x}_{n}\right) ,$$

the concentration field at each point may be assumed to be additive. As a consequence, a compound measurement ateach sensor location may be evaluated via summation of individual source contributions. A Gaussian noise (e.g., noise model 359) may be applied to the sensor measurement, with a standard deviation corresponding to the sensor’s systematic error, together with a detection threshold. For both error and threshold parameters, values reported by the sensor’s vendor of 0.002 ± 0.0001 ppmv over background level (estimated at 1.8 ppmv in the area of interest) may be used. Concentration values measured by each sensor may be relative to background concentration.

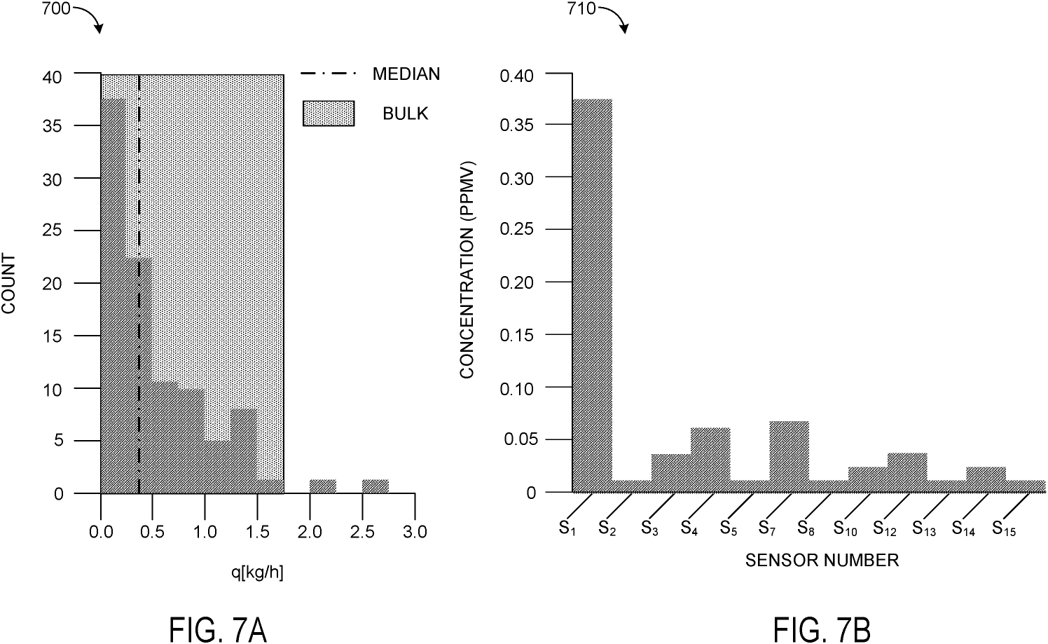

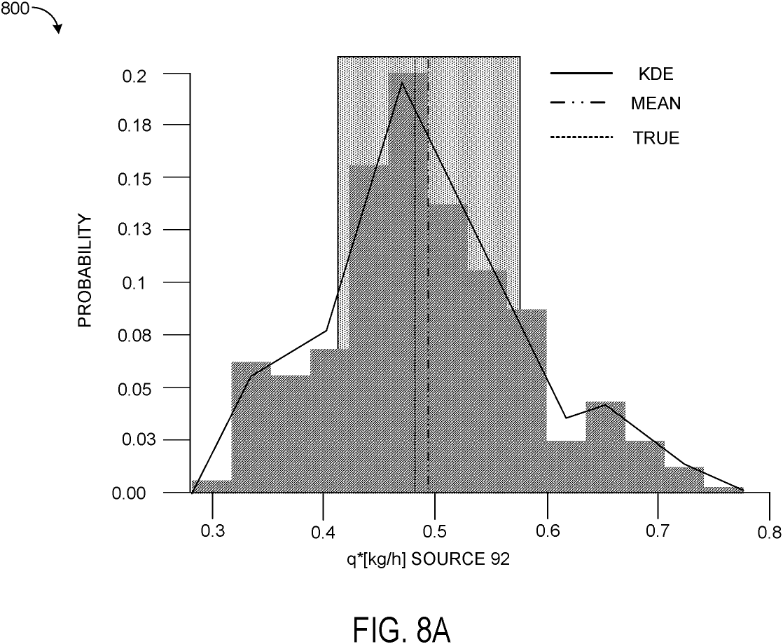

FIG. 7A depicts an example sample from an emission rate distribution, as a histogram 700, plotted together with median (dashed line) and bulk emission rates. The histogram of FIG. 7A represents an example pattern in which most sources have low emissions, with a few of the sources being super-emitters.

FIG. 7B depicts measurements of a concentration of a chemical species (e.g., methane), as a histogram 710, at sensor locations corresponding to the 15 sensors of FIG. 5, labeled as S₁ - S₁₅. As an example, the measurements of FIG. 7B may be generated by dispersion model 312, based on the emission rate distribution of FIG. 7A and the wind speed and angle distribution depicted in FIGS. 6A and 6B. In this example, only 12 of the 15 sensors exhibit above threshold measurements of the chemical species.

As an example of the optimization process, the Pyro implementation of the No-U-Turn, Hamiltonian Monte Carlo is used to sample the marginal posterior. In this example, a relatively small collection of 1000 samples provides a good compromise between accuracy and computational time. Sampling performed by a sampler returns the leak rate distribution for each of the 100 sources, at different degrees of convergence. As priors were obtained from empirical data, these are not necessarily conjugated, hence the marginal posterior distribution is unknown and needs to be fitted. Although it is possible to look for a continuous parametric fit, KDE 366 may be performed by the post processing component 330, for example, in scikit-learn using a grid search with cross validation to fix the kernel and bandwidth of each leak rate distribution.

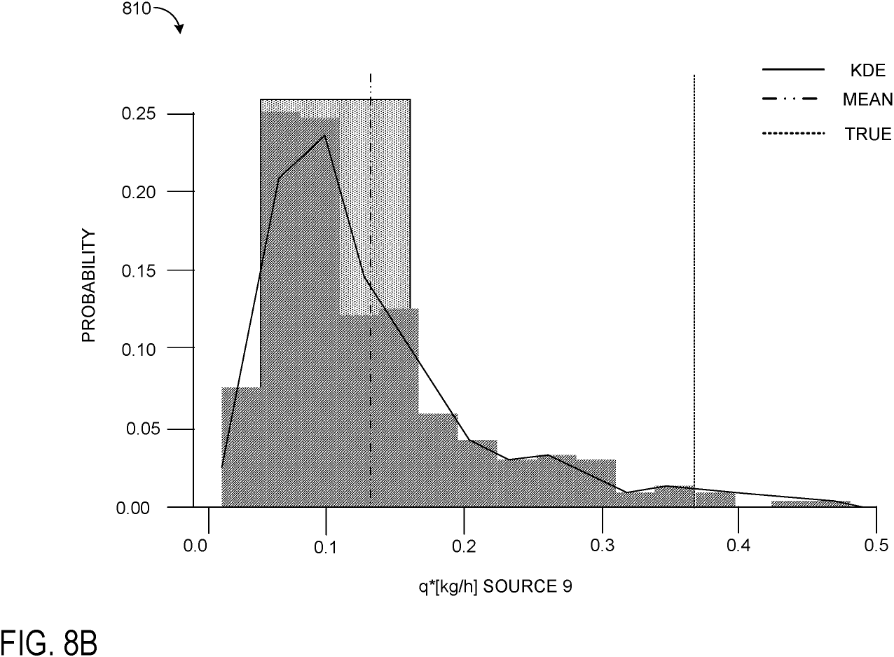

FIG. 8A depicts an example marginal posterior distribution 800 in which a samples histogram and a KDE fit are shown together with a 68% HPDI interval (shaded area), the sample mean, and the true value. In FIG. 8A, the sample mean provides a good estimation of the true value, as convergence has been achieved. Data representing the example marginal posterior distribution and the sample mean of FIG. 8A may be generated by source attribution module 300, as an example. However, it should be noted that the sample mean does not always track with the true value. FIG. 8B depicts another example marginal posterior distribution 810 where the true value lies in the tail of the distribution. In such examples, additional parametric fitting may be performed in order to achieve convergence before advancing.

As an example, 51 sample point estimates within the HPDI may be obtained, for each emission rate’s marginal posterior distributions. The posterior sample average, in this example, was found to be a robust central estimator. In addition, 50 percentiles points estimate (from 0 to 100 at steps of two) may be used. The point estimates may be used in the forward model (e.g., dispersion model 312) to estimate the predictive likelihood and the source contribution at each sensor, which may be used by the ranking model to extract the top three sources, per sensor, contributing the most to the measured methane concentration above baseline, together with their ranking confidence (e.g., Equation 13). The output from this process is 51 ranking and concentration values for each sensor.

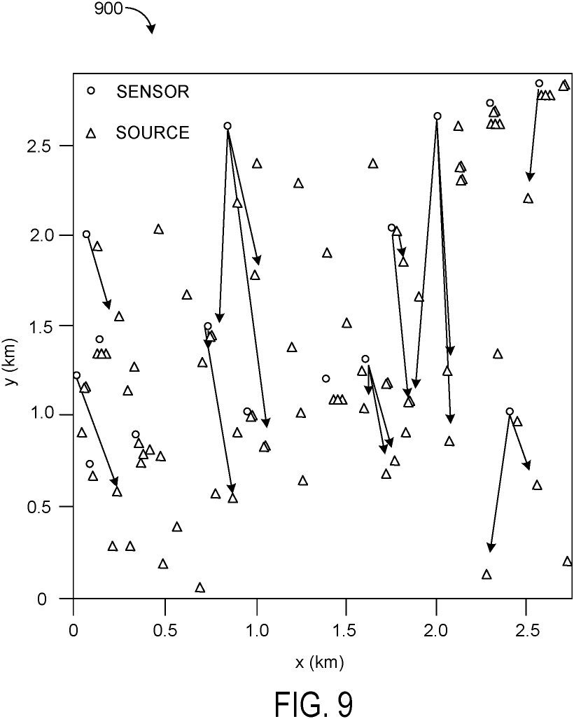

For each sensor, a maximum (predictive) likelihood value out of the 51 evaluated ranking and concentration values may be selected as the estimate for likely sources. FIG. 9 shows an example visualized of this result as a network map 900 showing sensor to source connectivity for the three selected sources per sensor, weighted by source emission rate. In at least some examples, this result constitutes the model recommendation presented to the monitoring operator, to help them plan further field investigation and plan emission remediation by prioritizing the most likely source of leakage.

A mean average precision at k=3 (mAP@3) was evaluated, as an example, which provides an indication of how many of the three proposed sources have been correctly ranked. The result may be averaged over all available sensors. In this example, mAP@3=0.86.

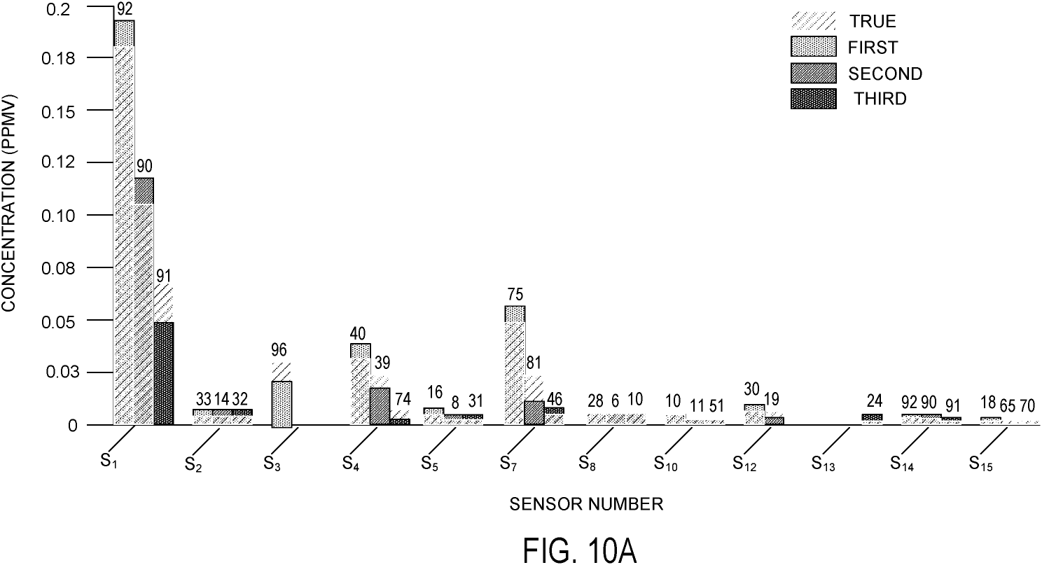

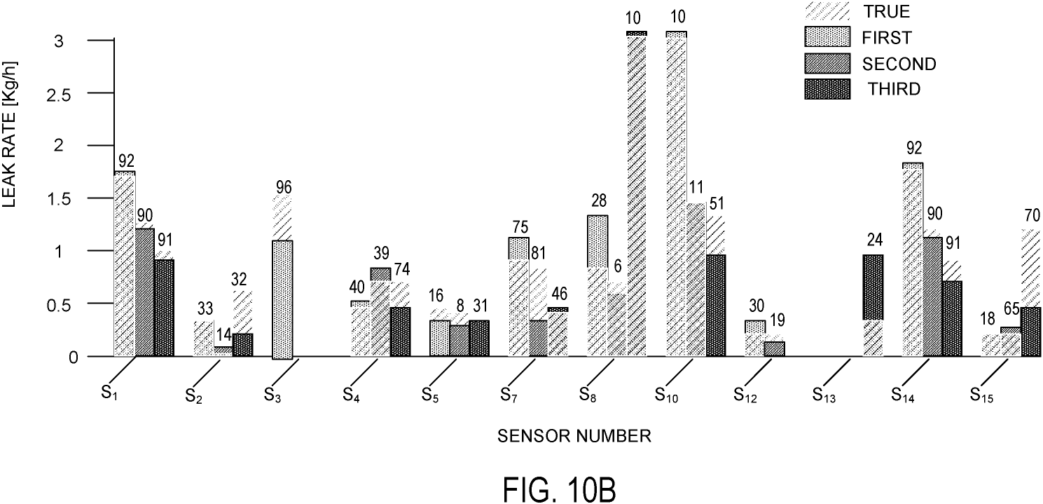

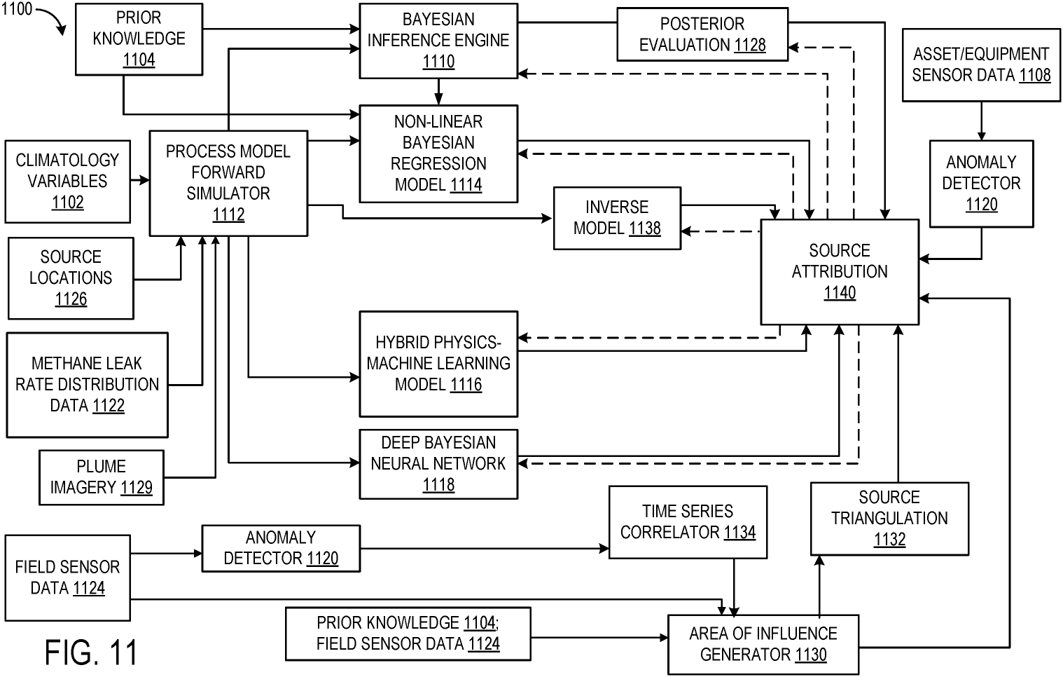

Ranking error may depend on the relative magnitude of the source’s emission rates. FIGS. 10A and 10B show the true and predicted source contributions to the methane concentration signal and emission rates detected at a sensor. FIG. 10A is a graph 1000 depicting an example of true (shaded) and predicted (solid) source contributions to a measurement of concentration (above background level) of a chemical species (e.g., methane), as a histogram, at each of the 15 sensors of FIG. 5. In this example, the three sources that contributed the most to the combined measurement at each sensor above the background level are identified above each bar of the histogram (e.g., sensor identifiers 1 - 100). For example, for sensor S₁, the three greatest contributors to the combined measurement of the chemical species above background level are sources 92, 90, and 91, respectively. The above analysis was repeated for additional days randomly sampled throughout a year at different times of the day. Depending on factors such as the weather, emission rate samples, and the number of sensors recording the concentration signal (as low as one sensor), the mAP@3 may vary, although on average is still ~ 0.83, showing the robustness of the model.

Emission rate quantification and source attribution may be outputs from the source attribution module implementing a Bayesian learning algorithm. Accurate emission size estimation may be used to quantify the environmental footprint of methane leaks. FIG. 10B is a graph 1010 depicting an example of true and predicted emission rate estimates for the highest three emission sources, as a histogram, at each of the 15 sensors of FIG. 5. In this example, the three sources with the highest predicted emission rate as depicted by each sensor are identified above each bar of the histogram (e.g., sensor identifiers 1 - 100).

Root Mean Squared Error (RMSE) may be used as a metric to evaluate the performance of the emission rate quantification algorithm. However, this compound error may not be representative, as some sources may be more difficult to estimate due to their lower emission rates and the skewed nature of the underlying distribution. As an illustrative example, medium emissions may have an RMSE of ~ 28%, compatible with the average error reported by aerial surveys, although the estimation is of a different nature, as no ground truth is known in the above example.

As an example, for high leak rate outliers, an RMSE ~ 4% was obtained, both for sources that have been correctly classified as contributing to the measured signal at a sensor (as determined by the mAP@3 metric). This emission rate estimate may also be used to update the prior leak rate distribution, which can then be used for future analyses. This may be thought of as a Bayesian learning process that iteratively improves the source attribution and estimation process.

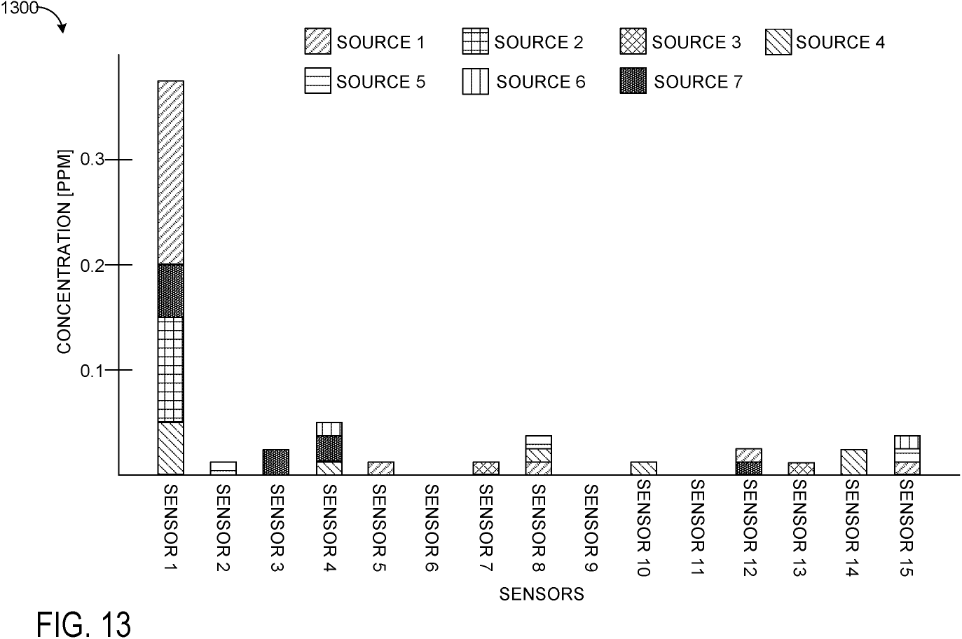

A source attribution framework implemented at source attribution module 242 of FIG. 2 may be highly customizable such that an end user can configure and utilize framework components that are most suitable for their scenario. For example, the described area of influence approach described further herein and with respect to FIG. 13 is not computationally expensive and can be run in a real-time manner; however, it only provides an approximate result. The Bayesian approaches described herein take into account variables not considered in the area of influence approach can therefore provide a more robust solution. However, the Bayesian approach may be more computationally intensive. The area of influence or Bayesian approaches may be selected and utilized depending upon whether real-time computation is required and/or whether an approximate result will suffice for the particular use case. Other technical benefits not specifically mentioned herein can also be realized through implementations of the disclosed subject matter.

FIG. 11 is a schematic architecture diagram showing aspects of the configuration and operation of a source attribution decision machine 1100 that may form part of source attribution module 242 of FIG. 2, and/or part of source attribution module 300 of FIG. 3, according to one embodiment. As will be described in greater detail below, the source attribution decision machine 1100 generates functionality for solving inverse problems, such as the source attribution (which might also be referred to as “source retrieval”) problem described above. In particular, the source attribution decision machine 1100 is configured to estimate the respective contributions, and locations if unknown, of the sources (e.g., emission sources 104-1, 104-2, 104-3, and 104-4) contributing to a compound signal.

According to various embodiments, the source attribution decision machine 1100 can leverage climatology variables 1102, prior knowledge 1104, such as prior data set 342 and/or data in the form of methane leak rate distribution data 1106 obtained from an aerial dataset such as NASA jet propulsion laboratory (JPL) aerial surveys in the Permian Basin or any other such sources of information, and data 1108 generated by sensors on assets and other equipment as input data. As illustrated in FIG. 11, the source attribution decision machine 1100 can also encompass a Bayesian inference engine 1110, a process model forward simulator 1112. Process model forward simulator 1112 may be an example of simulation component 310 of FIG. 3), a non-linear Bayesian regression model 1114 (as an example of non-linear Bayesian regression model 324 of FIG. 3), a hybrid physics-machine learning model 1116, a deep Bayesian neural network (BNN) 1118, and a machine learning-based univariate/multivariate anomaly detector 1120, which may be combined with a time series similarity technique such as dynamic time warping and “area of influence” computations to perform source triangulation. The components can be selected and configured according to a decision strategy and utilized to estimate the contributions and locations of emission sources contributing to a compound signal. Details regarding the configuration and operation of these components will be provided below.

It is to be appreciated that the components comprising the source attribution decision machine 1100 may be selected and configured based on various factors including, but not limited to, the availability of climatology variables 1102, methane leak rate distribution data 1122, data generated by sensors on assets 1108, field sensor data 1124, prior knowledge 1102, the quantity of computing resources (e.g., processor cycles) available or desired to be utilized, whether computations need to be performed in real-time or near real-time, and/or the desired precision of the output. The various components of the source attribution decision machine 1200 may be selected and configured based on other factors, or combinations of factors, in other embodiments.

As discussed briefly above, in one embodiment the source attribution decision machine 1100 is configured to find the decomposition of a compound signal to estimate the contribution of each emission source to the compound signal. In the case where the sources are sources of methane emissions, some or all of the field sensors deployed in a geographic locations such as that shown in FIG. 1 record field sensor data 1124 describing methane concentration signals (e.g., in parts per million) exceeding a determined threshold during some observation time.

As there are multiple sources being monitored in the field, each field sensor records a compound signal, that is assumed in some embodiments to be given by the linear combination of concentrations generated by a subset of sources at the location of the detection point. In this example, the source attribution decision machine 1100 is configured to find the decomposition of the compound signal to estimate the contribution of each source of methane.

In particular, the source attribution decision machine 1200 can be utilized to determine the k sources that contribute the most to the compound signal and provide an estimate of the strength of their contribution. In one embodiment, the source locations 1126 are known. In other embodiments, there are a mixture of known and unknown source locations.

In order to provide the functionality described above, the source attribution decision machine 1100 includes a Bayesian inference engine 1110 in some embodiments. The Bayesian inference engine 1110 combines information on the observed data (e.g., the field sensor data 1124 describing the methane concentration at the location of each field sensor), prior knowledge 1104 in the form of a prior distribution (e.g., methane leak rate distribution data 1122), and a likelihood function to produce a posterior probability distribution for each potential source location 1126.

According to various embodiments, the posterior probability can be evaluated at posterior evaluator 1128, which may be an example of posterior evaluator 332. For example, the posterior probability may be evaluated using a Markov Chain Monte Carlo (“MCMC”) algorithm, a Stochastic Variational Inference (“SVI”) algorithm, or another suitable algorithm. Model uncertainty may be due to experimental uncertainty on measured parameters and noise. It may thus be expressed by a density distribution of the estimated fitting parameters and the posterior distribution.

The source attribution decision machine 1100 also includes a process model forward simulator 1112 in some embodiments. The process model forward simulator 1112 includes a physics-based model, such as a Gaussian plume model, WRF-CHEM model, or a CALPUFF model. Using climatology variables 1102 as input, the physics-based model can calculate gas dispersion using the release rate, downwind, crosswind, vertical distances, and/or mean wind speed at the height of the release. The plume evolution over time (e.g., over a 24 hour period) can be utilized to determine the source locations 1126 and/or regions such that the likelihood of detecting a leak is maximized.

In some embodiments, a non-linear Bayesian regression model (“BRM”) 1114 fits the leak-rates using the output of the process model forward simulator 1112 and the previously observed measurements at the location of each sensor (e.g., prior knowledge 1104). In one embodiment, a custom Gaussian plume model is used as a forward model, wherein the likelihood is given by a multi-variate normal distribution centered on the residual between model output and observed concentration. Model performance in the under-parametrized regime can be enhanced using a sparsity-inducing kernel. This model assumes stationarity of the concentration field and related parameters over some time-scale depending on the scenario considered.

The source attribution decision machine 1100 also includes a hybrid physics-machine learning model (“HPML”) 1116 in some embodiments. The HPML 1116 may be a deep learning model trained on process model simulations, in one embodiment. The process model can be WRF-CHEM, CALPUFF, or another atmospheric model that can model the evolution of a methane plume over time.

The HPML 1116 uses physical constraints to select physical solutions out of the many possible solutions. In addition, methane leak rate distribution data 1122 and/or plume imagery 1129 such as, but not limited to, airborne and/or satellite visible/infrared imaging, spectrometer data, and/or plume imagery, can be utilized to add randomization into the HPML 1116. This approach is suitable both for forward and backward modeling; in the former case, it may provide a substantial speed improvement over traditional PDE solvers.

In some embodiments, model uncertainty is added to the HPML 1116 via the use of deep BNN 1118. In the source attribution decision machine 1100, the BNN 1118 provides uncertainty estimation for the non-linear, non-stationary model constrained by the HPML 1116 over the physical PDE domain. The advantages of this approach are twofold: it supplements the HPML 1116 forward model with uncertainty estimations that are not considered by commercial solvers such as CALPUFF; and also provides improved non-linear model estimation with respect to the simpler BRM 1114 that includes realistic time dependence.

In some embodiments, the source attribution decision machine 1100 also implements an area of influence generator 1130. The area of influence approach is a computationally inexpensive approach to identify potential emission sources corresponding to a detected methane sensor signal or methane concentration anomaly in a field location. In this approach, areas of influence (shown in FIG. 13) are computed for each time step, with sensor locations, wind direction, and wind speed as inputs, and potential sources located within that area are identified, and the field sensor data is enriched with these potential source location information.

In parallel, a workflow is triggered (in real-time), when the anomaly detector 1120, which can be implemented as a machine learning -based model for either univariate or multivariate anomaly detection, flags an “incident” in the field, say at time t. The flagged incident triggers a time series correlation computation, where methane sensors with “similar” signals within a search window Δt of the detected incident time are identified, even if these signals are not strong enough to warrant detection on their own.

Once the sensors generating similar signals have been identified, an area of influence can then be computed for each sensor, within which potential emission sources could be present. An area of overlap (e.g., source triangulation 1132) may then be computed in order to determine potential source locations more precisely than is possible with one sensor alone.

Supervisory control and data acquisition (SCADA) or asset sensor data 1108 can feed into either the Bayesian technique or the area of influence approach described above. SCADA system or asset sensor data 1108, when available, improves the confidence of estimates from any of the source retrieval techniques described herein.

In some embodiments, time series clustering and/or time series similarity can be performed by time series correlator 1134 on the output of anomaly detector 1120 to better inform the area of influence computations and to obtain more accurate estimates of the sources. Anomaly detector may detect an incident at a field sensor based on field sensor data 1124, and search a window Δt around the detected incident for similar readings at other sensors. The incident and the results of the search may then be provided to time series correlator 1134 for time correlation analysis. For example, in one embodiment, a dynamic time warping time series similarity algorithm is utilized. This algorithm allows computation of optimal alignment between two time series of different frequency, amplitude, and phase. This algorithm may provide better results than, for example, a simple autocorrelation measure or Euclidean distance measure because it accounts for varying time offsets at which a particular signal could appear in one sensor vs. another.

Source attribution decision machine 1100 implements an approach using an inverse model 1138 in order to create an adjoint source-sensor relationship. The source-sensor relationship may then be used to efficiently solve the advection-diffusion PDE by source attribution 1140. Source attribution 1140 may use two related approaches for generating leak rates for given sources. In a first scenario, where the position of all contributing sources is known, only leak rate quantification is performed for each contributing emission source. In a second scenario, there is a mixture of known and unknown source positions. In this scenario, both leak rate quantifications and partial source position identifications are performed. Source attribution 1140 may select locations for the placement of the unknown sources, and provide this information as feedback to iteratively refine solutions output by one or more of Bayesian inference engine 1110, non-linear Bayesian regression model 1114, hybrid physics-machine learning model 1116, deep Bayesian neural network 1118, posterior evaluation 1128, and inverse model 1138. Once previously unknown source locations have stabilized, leak rate quantifications for known and unknown sources may be generated, ranked and output, for example, as described with regard to FIGS. 3 and 4.

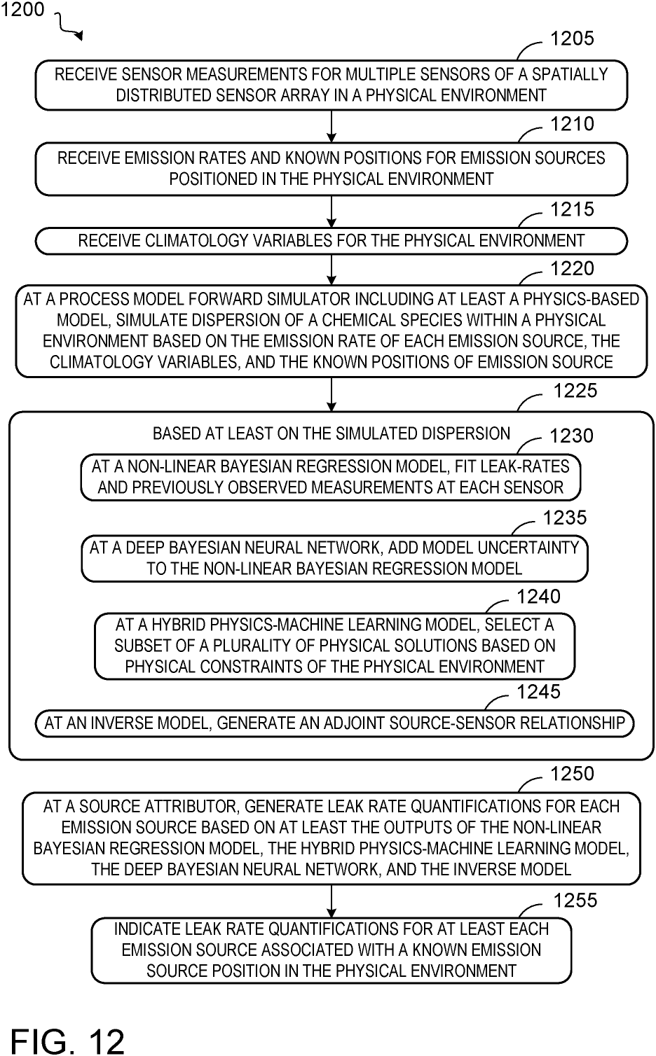

FIG. 12 is a flow diagram depicting an example method 1200 for a source attribution decision machine. Method 1200 may be performed by a computing system, such as example computing system 210 of FIG. 2. As an example, method 1200 may be performed by computing system 210 executing source attribution module 242, which may include any and/or all aspects of source attribution decision machine 1100.

At 1205, method 1200 includes receiving sensor measurements for multiple sensors of a spatially distributed sensor array in a physical environment. For example, sensor data 1108, field sensor data 1124 and prior knowledge 1104 may constitute examples of sensor measurements. At 1210, method 1200 includes receiving emission rates and known positions for emission sources positioned in the physical environment. For example, the source attribution decision machine may receive source locations 1126 and methane leak rate distribution data 1122. At 1215, method 1200 includes receiving climatology variables (e.g., climatology variables 1102, such as wind vector data) for the physical environment.

At 1220, method 1200 includes, at a process model forward simulator including at least a physics-based model, simulating dispersion of a chemical species within a physical environment based on the emission rate of each emission source, the climatology variables, and the known positions of emission source. For example, process model forward simulator 1112 may be used to simulate dispersion of a chemical species based on one or more dispersion models, such as a Gaussian plume dispersion model, WRF-CHEM model, an integrated Lagrangian puff modeling system, etc.

The simulated dispersion may then be output to one or more models for further analysis and modeling. For example, at 1225, method 1200 includes a plurality of modeling steps based at least on the simulated dispersion. At 1230, method 1200 includes, at a non-linear Bayesian regression model, fitting leak-rates and previously observed measurements at each sensor. For example, leak-rates of the chemical species may be fit based on the mapped concentration field and real-world, runtime measurements from sensors of the spatially distributed sensor array.

At 1235, method 1200 includes, at a deep Bayesian neural network, adding model uncertainty to the non-linear Bayesian regression model. For example, deep BNN 1118 may provide uncertainty estimations for a non-linear, non-stationary model constrained by HPML 1116 over the physical PDE domain. At 1240, method 1200 includes, at a hybrid physics-machine learning model, selecting a subset of a plurality of physical solutions based on physical constraints of the physical environment. For example, HPML 1116 may select physical solutions out of many possible physical solutions based on airborne and/or satellite imaging.

At 1245, method 1200 includes, at an inverse model, generating an adjoint source-sensor relationship. Such relationships may then be used to efficiently solve the advection-diffusion PDE by source attribution. At 1250, method 1200 includes, at a source attributor, generating leak rate quantifications for each emission source based on at least the outputs of the non-linear Bayesian regression model, the hybrid physics-machine learning model, the deep Bayesian neural network, and the inverse model. At 1255, method 1200 includes indicating leak rate quantifications for at least each emission source associated with a known emission source position in the physical environment.

Optionally, method 1200 may be configured to model emission sources with unknown locations in the physical environment. In such examples, method 1200 may include, at the source attributor, selecting locations in the physical environment for the emission sources with unknown locations. Method 1200 may further include providing the selected locations to one or more of the non-linear Bayesian regression model, the deep Bayesian neural network, the hybrid physics-machine learning model, and the inverse model. Method 1200 may additionally or alternatively include iteratively updating the leak rate quantifications at the source attributor based on at least the outputs of the non-linear Bayesian regression model, the hybrid physics-machine learning model, the deep Bayesian neural network, and the inverse model. Method 122 may further include indicating leak rate quantifications and selected locations in the physical environment for the emission sources with unknown locations

FIG. 13 is a plot diagram 1300 showing illustrative output generated by the source attribution decision machine 1100 shown in FIG. 11 according to one embodiment. As discussed briefly above, the output of the source attribution decision machine 1100 identifies the respective contributions of the sources contributing to a compound signal describing the emissions of a chemical species, such as methane. The example shown in FIG. 13, for instance, shows an example of the model output for 15 sensors and 100 sources. The illustrative plot 1300 indicates the source signal reconstruction for each measured compound signal at 12 of the 15 sensors (three sensors report no measurements).

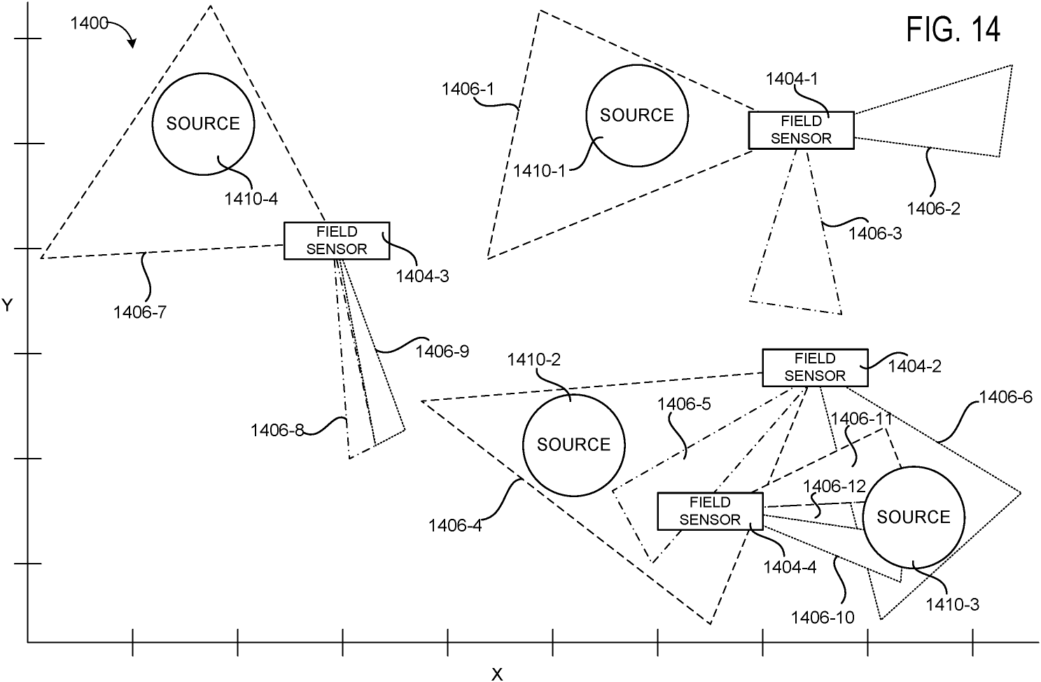

Turning to FIG. 14, a physical region 1400 is shown in X and Y dimensions four field sensors (1404-1, 1404-2, 1404-3, and 1404-4) are shown spatially positioned within physical region 1400. For each field sensor (1404-1, 1404-2, 1404-3, and 1404-4), one or more areas of influence (1406-1...1406-12) may be calculated, taking into account the sensor locations, wind direction, wind direction standard deviation, and/or wind speed at any point in time. For example, field sensor 1404-1 is shown associated with areas of influence 1406-1, 1406-2, and 1406-3; field sensor 1404-2 is shown associated with areas of influence 1406-4, 1406-5, and 1406-6; field sensor 1404-3 is shown associated with areas of influence 1406-7, 1406-8, and 1406-9; and field sensor 1404-4 is shown associated with areas of influence 1406-10, 1406-11, and 1406-12.

The combined areas of influence (1406-1...1406-12) may be used to compute one or more candidate areas of influence within which each potential emission source (1410-1, 1410-2, 1410-3, and 1410-4) could be located. For example, potential emission source 1410-1 is shown as being located within area of influence 1406-1; potential emission source 1410-2 is shown as being located within area of influence 1406-4; potential emission source 1410-3 is shown as being located within areas of influence 1406-6, 1406-10, and 1406-11; and potential emission source 1410-4 is shown as being located within area of influence 1406-7. Once candidate areas of influence have been computed, regions of overlap between areas of influence can be determined, and sources present in the overlapping areas can be deemed to be most likely to have contributed to a detected signal.

The process described above for identified areas of influence for each sensor may be used in conjunction with a source triangulation process in some embodiments. In these embodiments, an anomaly detector (e.g., anomaly detector 1120) can be implemented as a supervised or unsupervised machine learning algorithm that takes into account data describing routine emissions from a facility. The anomaly detector can identify whether a detected anomaly corresponds to an unintended emission scenario and return data indicating the severity of the event.

Once a leak has been detected in real-time, alerts can be triggered based on the severity of the unintended emission that can be sent to a monitoring dashboard. Root cause analysis (“RCA”) can then be performed, leveraging asset data from SCADA systems component to identify and isolate the offending methane emission source and enable operators to perform corrective measures.

A source attribution framework implemented at source attribution module 242 of FIG. 2 may be highly customizable such that an end user can configure and utilize framework components that are most suitable for their scenario. For example, the area of influence approach described herein and with regard to FIG. 14 is not computationally expensive and can be run in a real-time manner; however, it only provides an approximate result. The Bayesian approaches described herein and with regard to FIGS. 3, 4, 11, and 12 take into account variables not considered in the area of influence approach can therefore provide a more robust solution. However, the Bayesian approach may be more computationally intensive. The area of influence or Bayesian approaches may be selected and utilized depending upon whether real-time computation is required and/or whether an approximate result will suffice for the particular use case. Other technical benefits not specifically mentioned herein can also be realized through implementations of the disclosed subject matter.

While the subject matter described herein is presented primarily in the context of a source attribution decision framework configured for determining the strength of the contributions of a pollutant, such as methane, by multiple sources and the location of each source, if unknown, those skilled in the art will recognize that the source attribution decision framework disclosed herein can be used to estimate the contributions and locations of other types of sources to a compound signal. Those skilled in the art will also appreciate that the subject matter described herein can be practiced with various computer system configurations, including host computers in a distributed computing environment, hand-held devices, multiprocessor systems, microprocessor-based or programmable consumer electronics, computing or processing systems embedded in devices (such as wearable computing devices, automobiles, home automation etc.), minicomputers, mainframe computers, and the like.

In some embodiments, the methods and processes described herein may be tied to a computing system of one or more computing devices. In particular, such methods and processes may be implemented as a computer-application program or service, an application-programming interface (API), a library, and/or other computer-program product.

FIG. 15 schematically shows a non-limiting embodiment of a computing system 1500 that can enact one or more of the methods and processes described above. Computing system 1500 is shown in simplified form. Computing system 1500 may take the form of one or more personal computers, server computers, tablet computers, home-entertainment computers, network computing devices, gaming devices, mobile computing devices, mobile communication devices (e.g., smart phone), and/or other computing devices.

Computing system 1500 includes a logic machine 1510 and a storage machine 1520. Computing system 1500 may optionally include a display subsystem 1530, input subsystem 1540, communication subsystem 1550, and/or other components not shown in FIG. 15. Computing system 210 may be an example of computing system 1500. Logic machine 212 may be an example of logic machine 1510. Storage machine 214 may be an example of storage machine 1520. I/O subsystem 216 may be an example of input subsystem 1540 and/or communication subsystem 1550.

Logic machine 1510 includes one or more physical devices configured to execute instructions. For example, the logic machine may be configured to execute instructions that are part of one or more applications, services, programs, routines, libraries, objects, components, data structures, or other logical constructs. Such instructions may be implemented to perform a task, implement a data type, transform the state of one or more components, achieve a technical effect, or otherwise arrive at a desired result.

The logic machine may include one or more processors configured to execute software instructions. Additionally or alternatively, the logic machine may include one or more hardware or firmware logic machines configured to execute hardware or firmware instructions. Processors of the logic machine may be single-core or multi-core, and the instructions executed thereon may be configured for sequential, parallel, and/or distributed processing. Individual components of the logic machine optionally may be distributed among two or more separate devices, which may be remotely located and/or configured for coordinated processing. Aspects of the logic machine may be virtualized and executed by remotely accessible, networked computing devices configured in a cloud-computing configuration.

Storage machine 1520 includes one or more physical devices configured to hold instructions executable by the logic machine to implement the methods and processes described herein. When such methods and processes are implemented, the state of storage machine 1520 may be transformed-e.g., to hold different data.

Storage machine 1520 may include removable and/or built-in devices. Storage machine 1520 may include optical memory (e.g., CD, DVD, HD-DVD, Blu-Ray Disc, etc.), semiconductor memory (e.g., RAM, EPROM, EEPROM, etc.), and/or magnetic memory (e.g., hard-disk drive, floppy-disk drive, tape drive, MRAM, etc.), among others. Storage machine 1520 may include volatile, nonvolatile, dynamic, static, read/write, read-only, random-access, sequential-access, location-addressable, file-addressable, and/or content-addressable devices.

It will be appreciated that storage machine 1520 includes one or more physical devices. However, aspects of the instructions described herein alternatively may be propagated by a communication medium (e.g., an electromagnetic signal, an optical signal, etc.) that is not held by a physical device for a finite duration.

Aspects of logic machine 1510 and storage machine 1520 may be integrated together into one or more hardware-logic components. Such hardware-logic components may include field-programmable gate arrays (FPGAs), program- and application-specific integrated circuits (PASIC / ASICs), program- and application-specific standard products (PSSP / ASSPs), system-on-a-chip (SOC), and complex programmable logic devices (CPLDs), for example.

The terms “module,” “program,” and “engine” may be used to describe an aspect of computing system 1500 implemented to perform a particular function. In some cases, a module, program, or engine may be instantiated via logic machine 1510 executing instructions held by storage machine 1520. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc.

It will be appreciated that a “service”, as used herein, is an application program executable across multiple user sessions. A service may be available to one or more system components, programs, and/or other services. In some implementations, a service may run on one or more server-computing devices.

When included, display subsystem 1530 may be used to present a visual representation of data held by storage machine 1520. This visual representation may take the form of a graphical user interface (GUI). As the herein described methods and processes change the data held by the storage machine, and thus transform the state of the storage machine, the state of display subsystem 1530 may likewise be transformed to visually represent changes in the underlying data. Display subsystem 1530 may include one or more display devices utilizing virtually any type of technology. Such display devices may be combined with logic machine 1510 and/or storage machine 1520 in a shared enclosure, or such display devices may be peripheral display devices.

When included, input subsystem 1540 may comprise or interface with one or more user-input devices such as a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition; as well as electric-field sensing componentry for assessing brain activity.

When included, communication subsystem 1550 may be configured to communicatively couple computing system 1500 with one or more other computing devices. Communication subsystem 1550 may include wired and/or wireless communication devices compatible with one or more different communication protocols. As non-limiting examples, the communication subsystem may be configured for communication via a wireless telephone network, or a wired or wireless local- or wide-area network. In some embodiments, the communication subsystem may allow computing system 1500 to send and/or receive messages to and/or from other devices via a network such as the Internet.

In one example, a computer-implemented method for source attribution comprises, at a dispersion model, receiving measurements of a chemical species at multiple sensors of a spatially distributed sensor array in a physical environment for a given set of spatially positioned emission sources of the chemical species; based on the received measurements, mapping a concentration field of the chemical species from the set of emission sources to each of the multiple sensors using a forward operator; for each emission source, evaluating a likelihood data set that includes at least a set of sensor measurements, a variable set of source emission rates, and a parameter set for the physical environment, at least by fitting an emission rate of the chemical species using a regression model based on the mapped concentration field and real-world, runtime measurements from sensors of the spatially distributed sensor array; evaluating a posterior data set based at least on the evaluated likelihood data set and prior data for the physical environment; and for each sensor, determining an estimated emission rate and contribution ranking for each emission source based on the evaluation of the posterior data set; and outputting the determined estimated emission rates and contribution rankings for a predetermined number of highest ranking sources. In such an example, or any other example, the dispersion model is additionally or alternatively a physics-based dispersion model. In any of the preceding examples, or any other example, receiving measurements of the chemical species at multiple sensors of the spatially distributed sensor array in the physical environment for the given set of spatially positioned emission sources of the chemical species additionally or alternatively includes generating a distribution of sensor measurements based on a prior data set representing a distribution of a background emission rate of each source and a distribution of wind velocity in the physical environment. In any of the preceding examples, or any other example, forward operator based mapping is additionally or alternatively in the form of a non-linear, time-dependent mapping. In any of the preceding examples, or any other example, the regression model is additionally or alternatively a Bayesian regression model. In any of the preceding examples, or any other example, the Bayesian regression model is additionally or alternatively a non-linear Bayesian regression model. In any of the preceding examples, or any other example, the dispersion model is additionally or alternatively refined based on feedback from the Bayesian regression model. In any of the preceding examples, or any other example, evaluation of each posterior data set additionally or alternatively includes generating a kernel density estimation of the posterior data set; sampling the kernel density estimation of the posterior data set to obtain samples; and determining a probability-weighted emission rate based on the obtained samples. In any of the preceding examples, or any other example, the dispersion model is additionally or alternatively refined based on one or more of the obtained samples and the probability-weighted emission rate. The technical effect of implementing such a computer-implemented method is an improvement in pollutant detection accuracy and precision.

In another example, a system for determining source attribution, comprising a spatially distributed sensor array; a communication subsystem; a logic machine; and a storage machine holding instructions executable by a logic machine to execute a source attribution module, the source attribution module configured to: at a dispersion model, receive measurements of a chemical species at multiple sensors of the spatially distributed sensor array in a physical environment for a given set of spatially positioned emission sources of the chemical species; based on the received measurements, map a concentration field of the chemical species from the set of emission sources to each of the multiple sensors using a forward operator; for each emission source, evaluate a likelihood data set that includes at least a set of sensor measurements, a variable set of source emission rates, and a parameter set for the physical environment, at least by fitting an emission rate of the chemical species using a regression model based on the mapped concentration field and real-world, runtime measurements from sensors of the spatially distributed sensor array; evaluate a posterior data set based at least on the evaluated likelihood data set and historical data for the physical environment; and for each sensor: determine an estimated emission rate and contribution ranking for each emission source based on the evaluation of the posterior data set; and output the determined estimated emission rates and contribution rankings for a predetermined number of highest ranking sources. In such an example, or any other example, the dispersion model is additionally or alternatively a physics-based dispersion model. In any of the preceding examples, or any other example, receiving measurements of the chemical species at multiple sensors of the spatially distributed sensor array in the physical environment for the given set of spatially positioned emission sources of the chemical species additionally or alternatively includes generating a distribution of sensor measurements based on a prior data set representing a distribution of a background emission rate of each source and a distribution of wind velocity in the physical environment. In any of the preceding examples, or any other example, forward operator based mapping is additionally or alternatively in the form of a non-linear, time-dependent mapping. In any of the preceding examples, or any other example, the regression model is additionally or alternatively a Bayesian regression model. In any of the preceding examples, or any other example, the Bayesian regression model is additionally or alternatively a non-linear Bayesian regression model. In any of the preceding examples, or any other example, the dispersion model is additionally or alternatively refined based on feedback from the Bayesian regression model. In any of the preceding examples, or any other example, evaluation of each posterior data set additionally or alternatively includes generating a kernel density estimation of the posterior data set; sampling the kernel density estimation of the posterior data set to obtain samples; and determining a probability-weighted emission rate based on the obtained samples. In any of the preceding examples, or any other example, the dispersion model is additionally or alternatively refined based on one or more of the obtained samples and the probability-weighted emission rate. The technical effect of implementing such a system is an improvement in pollutant detection accuracy and precision.

In yet another example, a computer-implemented method for source attribution comprises receiving sensor measurements for multiple sensors of a spatially distributed sensor array in a physical environment; receiving emission rates and known positions for emission sources positioned in the physical environment; receiving climatology variables for the physical environment; at a process model forward simulator including at least a physics-based model, simulating dispersion of a chemical species within a physical environment based on the emission rate of each emission source, the climatology variables, and the known positions of emission source; based at least on the simulated dispersion, at a non-linear Bayesian regression model, fitting leak-rates and previously observed measurements at each sensor; at a deep Bayesian neural network, adding model uncertainty to the non-linear Bayesian regression model; at a hybrid physics-machine learning model, selecting a subset of a plurality of physical solutions based on physical constraints of the physical environment; and at an inverse model, generating an adjoint source-sensor relationship; at a source attributor, generating leak rate quantifications for each emission source based on at least the outputs of the non-linear Bayesian regression model, the hybrid physics-machine learning model, the deep Bayesian neural network, and the inverse model; and indicating leak rate quantifications for at least each emission source associated with a known emission source position in the physical environment. In such an example, or any other example, the method additionally or alternatively comprises for emission sources with unknown locations in the physical environment, at the source attributor, selecting locations in the physical environment for the emission sources with unknown locations; providing the selected locations to one or more of the non-linear Bayesian regression model, the deep Bayesian neural network, the hybrid physics-machine learning model, and the inverse model; iteratively updating the leak rate quantifications at the source attributor based on at least the outputs of the non-linear Bayesian regression model, the hybrid physics-machine learning model, the deep Bayesian neural network, and the inverse model; and indicating leak rate quantifications and selected locations in the physical environment for the emission sources with unknown locations. The technical effect of implementing such a computer-implemented method is an improvement in pollutant detection accuracy and precision.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A computer-implemented method for source attribution, comprising:
at a dispersion model, receiving measurements of a chemical species at multiple sensors of a spatially distributed sensor array in a physical environment for a given set of spatially positioned emission sources of the chemical species;
based on the received measurements, mapping a concentration field of the chemical species from the set of emission sources to each of the multiple sensors using a forward operator;
for each emission source, evaluating a likelihood data set that includes at least a set of sensor measurements, a variable set of source emission rates, and a parameter set for the physical environment, at least by fitting an emission rate of the chemical species using a regression model based on the mapped concentration field and real-world, runtime measurements from sensors of the spatially distributed sensor array;
evaluating a posterior data set based at least on the evaluated likelihood data set and prior data for the physical environment; and
for each sensor:
determining an estimated emission rate and contribution ranking for each emission source based on the evaluation of the posterior data set; and
outputting the determined estimated emission rates and contribution rankings for a predetermined number of highest ranking sources.

2. The method of claim 1, wherein the dispersion model is a physics-based dispersion model.

3. The method of claim 1, wherein receiving measurements of the chemical species at multiple sensors of the spatially distributed sensor array in the physical environment for the given set of spatially positioned emission sources of the chemical species includes generating a distribution of sensor measurements based on a prior data set representing a distribution of a background emission rate of each source and a distribution of wind velocity in the physical environment.

4. The method of claim 1, wherein forward operator based mapping is in the form of a non-linear, time-dependent mapping.

5. The method of claim 1, wherein the regression model is a Bayesian regression model.

6. The method of claim 5, wherein the Bayesian regression model is a non-linear Bayesian regression model.

7. The method of claim 5, wherein the dispersion model is refined based on feedback from the Bayesian regression model.

8. The method of claim 1, wherein evaluation of each posterior data set further includes:
generating a kernel density estimation of the posterior data set;
sampling the kernel density estimation of the posterior data set to obtain samples; and
determining a probability-weighted emission rate based on the obtained samples.

9. The method of claim 8, wherein the dispersion model is refined based on one or more of the obtained samples and the probability-weighted emission rate.

10. A system for determining source attribution, comprising:
a spatially distributed sensor array;
a communication subsystem;
a logic machine; and
a storage machine holding instructions executable by a logic machine to execute a source attribution module, the source attribution module configured to:
at a dispersion model, receive measurements of a chemical species at multiple sensors of the spatially distributed sensor array in a physical environment for a given set of spatially positioned emission sources of the chemical species;
based on the received measurements, map a concentration field of the chemical species from the set of emission sources to each of the multiple sensors using a forward operator;
for each emission source, evaluate a likelihood data set that includes at least a set of sensor measurements, a variable set of source emission rates, and a parameter set for the physical environment, at least by fitting an emission rate of the chemical species using a regression model based on the mapped concentration field and real-world, runtime measurements from sensors of the spatially distributed sensor array;
evaluate a posterior data set based at least on the evaluated likelihood data set and historical data for the physical environment; and
for each sensor:
determine an estimated emission rate and contribution ranking for each emission source based on the evaluation of the posterior data set; and
output the determined estimated emission rates and contribution rankings for a predetermined number of highest ranking sources.

11. The system of claim 10, wherein the dispersion model is a physics-based dispersion model.

12. The system of claim 10, wherein receiving measurements of the chemical species at multiple sensors of the spatially distributed sensor array in the physical environment for the given set of spatially positioned emission sources of the chemical species includes generating a distribution of sensor measurements based on a prior data set representing a distribution of a background emission rate of each source and a distribution of wind velocity in the physical environment.

13. The system of claim 10, wherein forward operator based mapping is in the form of a non-linear, time-dependent mapping.

14. The system of claim 10, wherein the regression model is a Bayesian regression model.

15. The system of claim 14, wherein the Bayesian regression model is a non-linear Bayesian regression model.

16. The system of claim 14, wherein the dispersion model is refined based on feedback from the Bayesian regression model.

17. The system of claim 10, wherein evaluation of each posterior data set further includes:
generating a kernel density estimation of the posterior data set;
sampling the kernel density estimation of the posterior data set to obtain samples; and
determining a probability-weighted emission rate based on the obtained samples.

18. The system of claim 17, wherein the dispersion model is refined based on one or more of the obtained samples and the probability-weighted emission rate.

19. A computer-implemented method for source attribution, comprising:
receiving sensor measurements for multiple sensors of a spatially distributed sensor array in a physical environment;
receiving emission rates and known positions for emission sources positioned in the physical environment;
receiving climatology variables for the physical environment;
at a process model forward simulator including at least a physics-based model, simulating dispersion of a chemical species within a physical environment based on the emission rate of each emission source, the climatology variables, and the known positions of emission source;
based at least on the simulated dispersion:
at a non-linear Bayesian regression model, fitting leak-rates and previously observed measurements at each sensor;
at a deep Bayesian neural network, adding model uncertainty to the non-linear Bayesian regression model;
at a hybrid physics-machine learning model, selecting a subset of a plurality of physical solutions based on physical constraints of the physical environment; and
at an inverse model, generating an adjoint source-sensor relationship;
at a source attributor, generating leak rate quantifications for each emission source based on at least the outputs of the non-linear Bayesian regression model, the hybrid physics-machine learning model, the deep Bayesian neural network, and the inverse model; and
indicating leak rate quantifications for at least each emission source associated with a known emission source position in the physical environment.

20. The method of claim 19, further comprising:
for emission sources with unknown locations in the physical environment, at the source attributor, selecting locations in the physical environment for the emission sources with unknown locations;
providing the selected locations to one or more of the non-linear Bayesian regression model, the deep Bayesian neural network, the hybrid physics-machine learning model, and the inverse model;
iteratively updating the leak rate quantifications at the source attributor based on at least the outputs of the non-linear Bayesian regression model, the hybrid physics-machine learning model, the deep Bayesian neural network, and the inverse model; and
indicating leak rate quantifications and selected locations in the physical environment for the emission sources with unknown locations.
